(19) United States
(12) Patent Application Publication
(10) Pub. No.: US 2022/0105127 A1
(43) Pub. Date: Apr. 7, 2022

(54) METHODS FOR PROVIDING THE BENEFITS OF METHIONINE RESTRICTION WITHOUT DIETARY RESTRICTION

(71) Applicant: Jamel Edmond Johnson, White Plains, UT (US)

(72) Inventor: Jamel Edmond Johnson, White Plains, UT (US)

(21) Appl. No.: 17/496,351

(22) Filed: Oct. 7, 2021

Related U.S. Application Data
(60) Provisional application No. 63/088,760, filed on Oct. 7, 2020.

(51) Int. Cl. A61K 33/04; A61K 31/198

(52) U.S. Cl. CPC A61K 33/04; A61K 31/198

(57) ABSTRACT

A method of providing a methionine restriction benefit to a subject without restricting the subject's diet is provided. The method includes administering an effective amount of a pharmaceutical composition to the subject. The pharmaceutical composition includes one or more of a first compound and a selenium compound. The first compound includes at least one compound chosen from lysine, O-benzyl serine (OBS), S-phenyl cysteine (SPC), S-benzyl cysteine (SBC), isoleucine, taurine, tryptophan, and 2-aminobicyclo-(2.2.1)-heptane-2-carboxylic acid (BCH). The second compound includes at least one compound chosen from selenomethionine, selenohomocysteine, selenocystathionine, selenocysteine/selenocystine, selenoglutathione trisulfide, methaneselenol, dimethyl selenide, trimethyl selenide, purified selenomethionine, selenized yeast extract, and pharmaceutically-acceptable salts of selenium-based acids.

### CROSS-REFERENCE TO RELATED APPLICATION

This application claims the benefit of U.S. Provisional Application No. 63/088,760 filed on Oct. 7, 2020.

### TECHNICAL FIELD

The disclosure relates to methods of providing improved health benefits for subjects that have diseases or disorders associated with diet-induced obesity, low metabolism, hyperinsulinemia, adipose tissue accumulation, fatty livers, and similar heath issues. More specifically, the disclosure relates to providing a subject with certain benefits associated with a methionine-restricted diet without having to restrict a subject's diets.

### BACKGROUND

Diseases or disorders associated with obesity, nutritional uptake, metabolism rates, and other diet-related diseases or disorders are a major health concern in the United States and other countries. Their complications may include hypertension, diabetes, coronary artery disease, stroke, congestive heart failure, venous disease, multiple orthopedic problems, and pulmonary insufficiency with markedly decreased life expectancy. As a result, according to the Centers for Disease Control and Prevention, these diseases and disorders are responsible for hundreds of thousands of deaths annually and cost the U.S. health care system an estimated $147 billion a year. The goal of addressing these diseases and disorders is to improve or prevent the morbidity and mortality presently associated with these types of diseases and disorders. Simply put, addressing these issues in a subject would improve the subject's health span.

Various approaches for treating these types of diseases and disorders include medical management or intervention such as psychotherapy, administration of medications, surgical procedures, and behavioral modification techniques. But these approached have not yielded exceptional results. One approach has been found to provide an improvement in a subject's health span, including reducing fat accumulation, improving metabolism, and hormone level control.

Dietary methionine restriction has been shown to improve mammalian health span. For example, rats fed a methionine-restricted diet are substantially longer-lived than their control-fed counterparts and show a marked amelioration of age-related pathologies. Among the varied benefits of a methionine-restricted to rodents, there has been shown an improvement in metabolic health, marked by reduced white adipose tissue accumulation, amelioration of liver steatosis, and improved glycemic control. Additionally, animals fed a high-fat diet meant to approximate the human Western diet have responded to methionine restriction in a way that demonstrates that this intervention protects against obesity and the health problems associated with obesity. In addition, as part of a multi-faceted response to dietarily restricted methionine intake, restricted animals also demonstrate altered plasma levels of the nutrient- and stress-sensing hormones IGF-1, FGF-21, adiponectin, and leptin. Studies have also suggested that the response to dietary methionine restriction is conserved throughout phylogeny and that dietary methionine restriction in humans is likely to produce similar health span benefits seen in rodents.

Methionine restriction in practice involves eating foods that are low in methionine. Though all protein has methionine, some protein sources are much lower in methionine than others. All animal sources (including milk and especially eggs) are high in methionine. A methionine-restricted diet may be considered a subset of a vegan diet given that the vegan diet is naturally low in proteins and free amino acids. Accordingly, a methionine-restricted diet is technically feasible for humans.

Although studies have suggested that humans (and other mammals) might benefit from dietary methionine restriction and that dietary methionine restriction is technically feasible for humans and other animals, there are challenges and disadvantages associated with adherence to such a diet, which renders widespread adherence problematic. One challenge to dietary methionine restriction is that limiting consumption of foods containing methionine also limits all of the other amino acids in that methionine-replete food. These other amino acids may be necessary to the body and limiting them might result in undesirable side effects. Additionally, a methionine-restricted diet requires voluntary dieting and studies have shown that voluntary dieting to achieve health benefits is mostly unsuccessful. Voluntary dieting is extremely difficult and most people simply do not have the willpower to limit the intake of food, or endure the physical, mental, or emotional challenges that often accompany voluntary dieting.

Accordingly, compositions and methods of providing the benefits associated with methionine-restriction are needed without requiring the dietary restriction of methionine intake. Compositions and methods are needed to produce health benefits that are similar to those produced by a reduction of methionine, but in the context of a subject's regular or normal methionine-replete diet.

### SUMMARY

Embodiments of the present invention include compositions and methods of providing the benefits of methionine restriction to a subject without restricting methionine intake through the subject's diet. A method includes administering an effective amount of a pharmaceutical composition or components thereof into the subject. In one embodiment, the pharmaceutical composition includes an effective amount of a first and/or second compound. The first compound may include one or more of lysine, tryptophan, O-benzyl serine (OBS), S-phenyl cysteine (SPC), S-benzyl cysteine (SBC), isoleucine, taurine, and 2-aminobicyclo-(2.2.1)-heptane-2-carboxylic acid (BCH). These first compounds, either alone or in combination with each other, may be used either alone or in combination with the second compound. In other embodiments, the pharmaceutical composition may only include the second compound.

The second compound may include selenium. In one embodiment, the selenium is organic selenium. In another embodiment, the selenium is inorganic selenium. In yet another embodiment, the selenium may be a combination of organic and inorganic selenium compounds and may include pharmaceutically-acceptable salts of acids containing selenium.

The method may include administering an effective amount of the pharmaceutical composition or components thereof to affect one or more of reduced adipose tissue accumulation, reduced body mass, reduced fatty liver, reduced circulating levels of IGF-1, increased circulating levels of FGF-21, reduced circulating levels of leptin, increased circulating levels of adiponectin, reduced circulating levels of glucose, reduced circulating levels of insulin and various combinations thereof.

Embodiments of the present invention also include compositions and methods of downregulating IGF-1 signaling in a subject. This method may include administering an effective amount of the pharmaceutical composition or components thereof described above into the subject. Embodiments described herein also describe compositions and methods for treating a disease or disorder associated with diet-induced obesity, including low metabolism, hyperinsulinemia, adipose tissue accumulation, fatty livers, diabetes, dermatitis, autoimmune disorders, allergies, rheumatoid arthritis, asthma, endometriosis, inflammatory bowel disease, glomerulonephritis, hepatitis, coeliac disease, ischemia-reperfusion injury, transplant rejection, or combinations of these in a subject. Embodiments of this method may include administering an effective amount of the pharmaceutical composition or components thereof described above. Embodiments of the present invention also may include using the pharmaceutical composition described herein in the manufacture of a medicament for providing reduced adipose tissue accumulation, reduced body mass, reduced fatty liver, reduced circulating levels of IGF-1, increased circulating levels of FGF-21, reduced circulating levels of leptin, increased circulating levels of adiponectin, reduced circulating levels of glucose, and/or reduced circulating levels of insulin, or the manufacture of a medicament for treating a disease or disorder associated with diet-induced obesity, low metabolism, hyperinsulinemia, adipose tissue accumulation, fatty livers, diabetes, dermatitis, autoimmune disorders, allergies, rheumatoid arthritis, asthma, endometriosis, inflammatory bowel disease, glomerulonephritis, hepatitis, coeliac disease, ischemia-reperfusion injury, transplant rejection, or combinations thereof in a subject.

Accordingly, embodiments described herein describe compositions and methods to accomplish the benefits associated with dietary methionine restriction by supplementation and/or intervention, not dietary restriction.

### BRIEF DESCRIPTION OF THE DRAWINGS

FIG. 1 shows a bar graph the reduced weight gain of male mice supplemented with sodium selenite (SS) and methionine-restricted (MR) littermates as compared with untreated control animals, all groups having been fed an otherwise normal high-fat diet ad libitum;

FIG. 2A shows a bar graph depicting the reduced inguinal adipose tissue accumulation of male mice supplemented with sodium selenite (SS) and methionine-restricted (MR) littermates as compared with untreated control animals, all groups having been fed an otherwise normal high-fat diet ad libitum;

FIG. 2B shows a bar graph depicting the reduced perigonadal adipose tissue accumulation of male mice supplemented with sodium selenite (SS) and methionine-restricted (MR) littermates as compared with untreated control animals, all groups having been fed an otherwise normal high-fat diet ad libitum;

FIG. 3A shows a bar graph depicting the longitudinal maintenance of glucose homeostasis in male mice supplemented with sodium selenite (SS) and methionine-restricted (MR) littermates as compared with untreated control animals, all groups having been fed an otherwise normal high-fat diet ad libitum;

FIG. 3B shows a bar graph depicting the longitudinal maintenance of insulin homeostasis in male mice supplemented with sodium selenite (SS) and methionine-restricted (MR) littermates as compared with untreated control animals, all groups having been fed an otherwise normal high-fat diet ad libitum;

FIG. 4A shows a bar graph depicting the longitudinal maintenance of low IGF-1 levels in male mice supplemented with sodium selenite (SS) and methionine-restricted (MR) littermates as compared with untreated control animals, all groups having been fed an otherwise normal high-fat diet ad libitum;

FIG. 4B shows a bar graph depicting the longitudinal maintenance of low leptin levels in male mice supplemented with sodium selenite (SS) and methionine-restricted (MR) littermates as compared with untreated control animals, all groups having been fed an otherwise normal high-fat diet ad libitum;

FIG. 5 shows a bar graph the depicting the reduced weight gain of female mice supplemented with S-phenyl-cysteine (SPC) and methionine-restricted (MR) littermates as compared with untreated control animals, all groups having been fed an otherwise normal high-fat diet ad libitum;

FIG. 6A shows a bar graph depicting the reduced inguinal adipose tissue accumulation of female mice supplemented with S-phenyl-cysteine (SPC) and methionine-restricted (MR) littermates as compared with untreated control animals, all groups having been fed an otherwise normal high-fat diet ad libitum;

FIG. 6B shows a bar graph depicting the reduced perigonadal adipose tissue accumulation of female mice supplemented with S-phenyl-cysteine (SPC) and methionine-restricted (MR) littermates as compared with untreated control animals, all groups having been fed an otherwise normal high-fat diet ad libitum;

FIG. 7A shows a plot graph depicting the longitudinal maintenance of glucose homeostasis in female mice supplemented with S-phenyl-cysteine (SPC) and methionine-restricted (MR) littermates as compared with untreated control animals, all groups having been fed an otherwise normal high-fat diet ad libitum.

FIG. 7B shows a plot graph depicting the longitudinal maintenance of insulin homeostasis in female mice supplemented with S-phenyl-cysteine (SPC) and methionine-restricted (MR) littermates as compared with untreated control animals, all groups having been fed an otherwise normal high-fat diet ad libitum.

FIG. 8A shows a bar graph depicting the longitudinal maintenance of low IGF-1 levels in female mice supplemented with S-phenyl-cysteine (SPC) and methionine-restricted (MR) littermates as compared with untreated control animals, all groups having been fed an otherwise normal high-fat diet ad libitum;

FIG. 8B shows a bar graph depicting the longitudinal maintenance of low leptin levels in female mice supplemented with S-phenyl-cysteine (SPC) and methionine-restricted (MR) littermates as compared with untreated control animals, all groups having been fed an otherwise normal high-fat diet ad libitum;

FIG. 9A shows a bar graph depicting the reduced inguinal adipose tissue accumulation of male mice supplemented with selenomethionine (SMS) and methionine-restricted (MR) littermates as compared with untreated control animals, all groups having been fed an otherwise normal low-fat diet ad libitum; and

FIG. 9B shows a bar graph depicting the reduced perigonadal adipose tissue accumulation of male mice supplemented with selenomethionine (SMS) and methionine-restricted (MR) littermates as compared with untreated control animals, all groups having been fed an otherwise normal low-fat diet ad libitum.

### DETAILED DESCRIPTION

In the following detailed description, reference is made to the accompanying drawings which form a part hereof. The detailed description includes various embodiments of the compositions and methods of the present invention. These embodiments are described in sufficient detail to enable those of ordinary skill in the art to practice the disclosure. However, before the present materials and methods are described, it is to be understood that this invention is not limited to the particular molecules, compositions, active ingredients, methodologies, or protocols herein described, as these may vary in accordance with routine experimentation and optimization. It is also to be understood that the terminology used in the description is for the purpose of describing the particular versions or embodiments only and is not intended to limit the scope of the embodiments described herein. Accordingly, various substitutions, modifications, additions, rearrangements, or combinations thereof are within the scope of this disclosure. Furthermore, all or a portion of any embodiment disclosed herein may be utilized with all or a portion of any other embodiment, unless stated otherwise.

The section headings provided herein are for convenience only and do not interpret the scope or meaning of the claimed options. Furthermore, unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

### Definitions

As used herein and in the appended claims, the singular forms “a”, “an,” and “the” include plural reference unless the context clearly dictates otherwise.

The terms “A or B,” “at least one of A and B,” “one or more of A and B”, or “A and/or B” as used herein include all possible combinations of items enumerated with them. For example, use of these terms, with A and B representing different items, means: (1) including at least one A; (2) including at least one B; or (3) including both at least one A and at least one B. In addition, the articles “a” and “an” as used herein should generally be construed to mean “one or more” unless specified otherwise or clear from the context to be directed to a singular form.

The expression “configured to” as used herein may be used interchangeably with “suitable for,” “having the capacity to,” “designed to,” “adapted to,” “made to,” or “capable of” according to a context. The term “configured” does not necessarily mean “specifically designed to,” and the expression compound or composition “configured to . . . ” may mean that the compound or composition is, either alone or in combination with other compounds or compositions, configured to produce or effect a certain desired result.

Unless the context otherwise requires, in the description text and in the claims that follow, the term “contain” and its derivatives, such as “contains” and “containing,” should be considered open, non-restrictive forms, that is, as “including but not limiting.” In addition, the terms “having,” or “including” should be understood as “including but not limited to the specific member or members listed.” Thus, a compound or composition that “contains” or “includes” selenium and/or other compounds as an active ingredient, for example, may have additional active ingredients, compounds, or compositions.

The term “about,” as used herein, includes any value that is within 10% of the described value.

The term “between,” as used herein, is inclusive of the lower and upper number of the range.

Reference herein to any numerical range (for example, a dosage range) expressly includes each numerical value (including fractional numbers and whole numbers) encompassed by that range. For example, but without limitation, reference herein to a range of 0.5 mg/dL to 100 mg/dL explicitly includes all whole numbers and fractional numbers between the two.

As used herein, the terms “administration” and “administering” refer to the act of providing a therapeutic, prophylactic, or other agent to a subject for the treatment or prevention of one or more diseases or disorder. Exemplary routes of administration to the human body are through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the term “disease or disorder” includes any and all conditions, symptoms, and/or effects that may be associated with the disease or disorder.

As used herein, the term “pharmaceutical composition” refers to an agent or active ingredient (e.g., a selenium compound and/or other compound) with or without a carrier, excipient, or other ingredients or impurities, whether inert or active, making the composition suitable for diagnostic or therapeutic use in vitro, in vivo or ex vivo. A “pharmaceutical composition” may be embodied in a medicament, therapeutic, supplement, and the like. The term “pharmaceutical composition” is not limited to compositions needing a prescription to be administered, but includes compositions that may be embodied in forms that don't need a prescription to be used or otherwise obtained.

The terms “subject,” “host,” and “patient” refer to an animal, including, but not limited to, a primate (e.g., human), cow, pig, sheep, goat, horse, dog, cat, rabbit, rat, or mouse that is studied, analyzed, tested, diagnosed, or treated. The terms “subject,” “host,” and “patient” are used interchangeably herein, unless indicated otherwise. In certain embodiments, the subject has or is susceptible to having a disease or disorder provided herein.

The use of “diet” when used in conjunction with a subject may refer to the normal or typical diet, including without limitation, the caloric and/or nutritional consumption of the particular subject. The “diet” of a subject may also refer to a recommended daily allowance of calories or nutrients for the particular age, height, and weight of a particular subject.

The terms “treat,” “treatment,” or “treating”, as used herein in any tense, refer to any method used to partially or completely alleviate, ameliorate, relieve, inhibit, prevent, delay onset of, reduce severity of and/or reduce incidence of one or more symptoms, conditions, or features of a disease or disorder. Treatment may be administered to a subject who does not exhibit signs of a disease or disorder. In some embodiments, treatment may be administered to a subject who exhibits only early signs of the disease or disorder for the purpose of decreasing the risk of developing pathology associated with the disease or disorder. It will be appreciated that, although not precluded, treating a disease or disorder does not require that the disease, disorder, or conditions or symptoms associated therewith be completely eliminated. A subject that is administered a pharmaceutical composition or constituents thereof is deemed to have been treated with the pharmaceutical composition or constituents thereof.

The terms “prevent,” “preventing,” or “prevention,” as used herein, include inhibiting or preventing a disease or disorder and any conditions, symptoms, or effects thereof as well as preventing or inhibiting the underlying causes of such diseases, disorders, conditions, symptoms, e.g., arresting the development of the disease or disorder and are intended to include prophylaxis. The terms further include achieving a prophylactic benefit. For prophylactic benefit, the compositions are optionally administered to a patient at risk of developing a particular disease or disorder to a subject reporting one or more of the physiological symptoms of a disease, disorder, or condition or to a subject at risk of reoccurrence of the disease or disorder.

The terms “effective amount” or “therapeutically effective amount,” as used herein, refer to a sufficient amount of at least one agent, compound, or compositions being administered which achieve a desired result, e.g., to provide to some extent one or more health benefits or to relieve to some extent one or more symptoms of a disease or disorder being treated. In certain instances, the result is a reduction and/or alleviation of the signs, symptoms, or causes of a disease, or any other desired alteration of a biological system. In certain instances, an “effective amount” for therapeutic uses is the amount of the agent at dosages and for periods of time necessary to achieve the desired therapeutic or prophylactic result. As will be apparent to those skilled in the art, it is to be expected that the effective amount of an agent, compound, or composition disclosed herein may vary according to factors such as the disease state, age, sex, and weight of the individual, and the ability of the pharmaceutical composition or composition to elicit a desired response in the particular individual. An “effective amount” or “therapeutically effective amount” is also one in which any toxic or detrimental effects of the treatment agent, compound, or composition are outweighed by the therapeutically beneficial effects.

The term “pharmaceutically-acceptable,” as used herein, refers to a material that does not abrogate the biological activity or properties of the agents, compounds, or compositions described herein, does not substantially produce adverse, allergic, or immunological reactions when administered to a subject, and is relatively non-toxic (i.e., the toxicity of the material significantly outweighs the benefit of the material). In some instances, a pharmaceutically-acceptable material may be administered to an individual without causing significant undesirable biological effects or significantly interacting in a deleterious manner with any of the components of the composition in which it is contained.

As used herein, “pharmaceutically-acceptable salts” refer to derivatives of the pharmaceutical composition or composition wherein the pharmaceutical composition or composition is modified by reacting it with an acid or base as needed to form an ionically bound pair. Examples of pharmaceutically-acceptable salts include conventional non-toxic salts or the quaternary ammonium salt of the parent compound formed, for example, from non-toxic inorganic or organic acids. Suitable non-toxic salts include those derived from inorganic acids such as hydrochloric, hydrobromic, sulfuric, sulfamic, phosphoric, nitric, and others known to those of ordinary skill in the art. The salts prepared from organic acids such as amino acids, acetic, propionic, succinic, glycolic, stearic, lactic, malic, tartaric, citric, ascorbic, pamoic, maleic, hydroxymaleic, phenylacetic, benzoic, salicylic, sulfanilic, fumaric, oxalic, isethionic, and others known to those of ordinarily skilled in the art. List of other suitable salts are found in Remington's Pharmaceutical Sciences, 17th edition. Mack Publishing Company, Easton Pa., 1985. p. 1418, the relevant disclosure of which is hereby incorporated by reference.

The term “carrier,” as used herein, refers to relatively non-toxic chemical agents that, in certain instances, facilitate the incorporation of an agent into cells or tissues. “Carriers” may include, but are not limited to, any of the standard pharmaceutical carriers such as a phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintegrants (e.g., potato starch or sodium starch glycolate), and the like.

As used herein, “pharmaceutically-acceptable carrier” includes any material which, when combined with a compound or composition of the invention, allows the compound or composition to retain biological activity, such as the ability to treat the associated disease or affect the various mechanisms associated therewith, and is non-reactive with the subject's immune system.

“Pharmaceutically-acceptable excipients,” as used herein, include but are not limited to binders, diluents, lubricants, disintegrants, glidants, and surface-active agents. Such “excipients” include carbohydrate or protein fillers, such as sugars, including lactose, sucrose, mannitol, or sorbitol. “Excipients” also include cornstarch, wheat, rice, potato, cellulose, and gums.

A “therapeutically effective dose” refers to the amount that provides a benefit, including improving or preventing symptoms of a disease or disorder.

The terms “methionine restriction-like state,” “MR State,” or “MRS” include any state within a subject that provides the health benefits of the diet known as methionine restriction to the subject, including, without limitation, protection of humans and mammalian animals from accumulating adipose tissue, liver steatosis, metabolic syndrome, and/or providing any other methionine restriction benefit.

The terms “first compound” or “methionine competitor” means any compound that is responsible for the inhibition of the transport activity by the different amino acid transporter mechanisms responsible for methionine uptake or that otherwise counteracts the metabolic effects of methionine without inhibiting amino acid transport. Regarding inhibition of transport activity, it is not limited to the apical or luminal membranes in the digestive track but may occur in any transport mechanism relating to methionine uptake wherever it may be found. Further, inhibition of transport activity by the different amino acid transporter mechanisms responsible for methionine uptake is not limited to saturation of any particular binding site for any particular amino acid transporter mechanism. Inhibition of transport activity by the different amino acid transporter mechanisms responsible for methionine uptake includes any method of reducing or eliminating the rate, efficiency, availability, or ability of methionine uptake.

The term “selenium,” as used herein throughout, includes the mineral selenium and compounds, compositions, or other materials that contain or include selenium in any of its forms, derivatives, and/or analogs. Such selenium forms include without limitation, selenates, selenites, selenides, and the like. Further, the term “selenium compound” means any compound, composition, or material that includes selenium. Indeed, references to materials that are “selenium-based” are meant to include materials that contain selenium, and vice versa. “Selenium” and “selenium compound” may be used interchangeably herein to denote the same thing.

Similarly, the terms “organoselenium” (sometimes referred to herein as “organic selenium”) and “inorganic selenium” include compounds, compositions, or other materials that contain or include organic selenium and inorganic selenium, respectively, in any of their respective forms, derivatives, and/or analogs.

The term “methionine-replete diet” in reference to a subject means that the subject has not had their methionine intake or consumption purposefully reduced by limiting the intake of things containing methionine. This term does not necessarily mean that the subject is getting a recommended amount of methionine in their diet. A subject's diet may be said to be “methionine-replete” without the subject achieving a certain minimum level of methionine intake, so long as the subject's diet has not been altered to restrict or reduce levels of methionine intake or consumption.

### Methionine Restriction Benefits

The restriction of methionine has been shown to provide many benefits, including, without limitation, improving metabolic health, reducing white adipose tissue accumulation, ameliorating liver steatosis, and improving glycemic control. Other methionine restriction benefits include protection against diet-induced obesity. Other methionine restriction benefits may include the starvation of certain cancer cells. In one embodiment, composition and methods of the present invention may provide one or more methionine restriction benefits including, without limitation, reduced adipose tissue accumulation, reduced body mass, reduced fatty liver, reduced circulating levels of IGF-1, increased circulating levels of FGF-21, reduced circulating levels of leptin, increased circulating levels of adiponectin, reduced circulating levels of glucose, reduced circulating levels of insulin, and/or combinations thereof.

References herein throughout to methods for providing a methionine restriction benefit are meant to include or encompass treating a subject lacking the particular methionine restriction benefit. By way of non-limiting example, a method of providing the methionine restriction benefit of reduced adipose tissue accumulation in a subject includes treating a subject, or providing a pharmaceutical composition to a subject, in order to reduce adipose tissue accumulation in the subject. Similarly, a method for providing the methionine restriction benefit of reduced fatty liver in a subject includes treating a subject, or providing a pharmaceutical composition to a subject, in order to reduce fatty liver in the subject. The forgoing correlation examples apply equally to all methionine restriction benefits and combinations of benefits. Thus, as used herein throughout, providing a methionine benefit to a subject is the same as treating a subject to effect that benefit and/or providing a pharmaceutical composition to the subject to effect that benefit.

### Pharmaceutical Compositions

Embodiments of the present invention teach how to achieve a methionine restriction-like state metabolically, providing one or more of these methionine restriction benefits without dietarily restricting methionine consumption. Embodiments of the present invention describe the achievement of methionine restriction benefits with a methionine-replete diet. This may be accomplished through the administration of, or treatment with, a pharmaceutical composition that includes non-selenium and/or selenium compounds. As used herein throughout, the terms “pharmaceutical composition” and “effective amount of a pharmaceutical composition” may be used interchangeably to denote a pharmaceutical composition that contains an effective amount of at least a non-selenium or “first compound,” an effective amount of at least selenium or a “selenium compound,” or at least an effective amount of both a first compound and a selenium compound.

Non-Selenium Pharmaceutical Compositions

In one embodiment, a pharmaceutical composition includes a first compound chosen from one or more of lysine, O-benzyl serine (OBS), S-phenyl cysteine (SPC), S-benzyl cysteine (SBC), isoleucine, taurine, tryptophan, and 2-aminobicyclo-(2.2.1)-heptane-2-carboxylic acid (BCH). These compounds, either alone or in combination with each other, or in combination with other compounds described herein, may affect certain metabolic pathways within a subject to achieve a methionine restriction-like state and the accompanying benefits thereof, without dietarily restricting the methionine consumption of the subject receiving the pharmaceutical composition.

In embodiments where the pharmaceutical composition includes the first compound, the first compound may be a methionine transport inhibitor where the first compound inhibits methionine consumed by a subject from being absorbed into the subject's blood stream from the subject's gut. Thus, a methionine restriction-like state can be achieved without limiting the subject's methionine consumption. In certain embodiments of this pharmaceutical composition, the first compound may inhibit methionine from being absorbed from the subject's stomach, upper intestine or lower intestine into the subject's bloodstream. The methionine transport inhibitor may inhibit the 1° methionine transporter B° AT1 from transporting methionine ingested or found within the subject from transporting from the subject's gut into the subject's bloodstream. It will be appreciated that certain methionine transport receptors may only be found in specific places within the gut or digestive track. Accordingly, depending upon the particular first compound in the pharmaceutical composition, the pharmaceutical composition, or compositions containing the pharmaceutical composition, may be configured to deliver the first compound to a particular place within the gut. By way of non-limiting example, the pharmaceutical composition, or compositions containing the pharmaceutical composition may be configured with a time release or other carrier to be delivered to the stomach, small or large intestine, or other places where a methionine transport receptor may reside. In other embodiments, a pharmaceutical composition that includes the first compound need not act as a methionine transport inhibitor.

A pharmaceutical composition used to treat or be administered to a subject may contain a first compound in an amount that is greater than about 2.5 times the amount of sulfur-containing amino acids in the subject's diet by weight. In other embodiments, the amount of first compound in a pharmaceutical composition may be greater than about 5 times the amount of sulfur-containing amino acids in the subject's diet by weight. In yet other embodiments, an amount of first compound in a pharmaceutical composition administered to a subject may be less than about 7.5 times the amount of sulfur-containing amino acids in the subject's diet. In yet other embodiments, an amount of first compound in a pharmaceutical composition administered to a subject may be less than about 10 times the amount of sulfur-containing amino acids in the subject's diet.

The average American daily diet contains about 2.4 grams of sulfur amino acids. (See www.sciencedirect.com/topics/medicine-and-dentistry/sulfur-amino-acid, citing Jason M. Hansen, Dean P. Jones, in Nutritional Oncology (Second Edition), 2006, Chapter 15). In one embodiment, a daily dose amount of first compound in a pharmaceutical composition for administration to a subject may be about 2.4 g times 2.5, or about 6 grams. In another embodiment, a daily dose amount of first compound contained in a pharmaceutical composition for administration to a subject may be about 2.4 g times 5, or about 12 grams. In another embodiment, a daily dose amount of first compound to be administered to a subject by way of a pharmaceutical composition may be about 2.4 g times 7.5, or about 18 grams. In another embodiment, a daily dose amount of first compound may be about 2.4 g times 10, or about 24 grams.

Sulfur amino acid intake ranges from between about 0.5 g to 5 g, inclusive. (See, Id.). In one embodiment, an effective amount of first compound in a pharmaceutical composition for administration to a subject may be greater than about 0.5 g times 2.5, or about 1.25 grams. In another embodiment, an amount of first compound in a pharmaceutical composition for administration to a subject may be greater than about 0.5 g times 5, or about 2.5 grams. In yet another embodiment an amount of first compound in a pharmaceutical composition for administration to a subject may be less than about 5 g times 7.5, or about 37.5 grams. In yet another embodiment an amount of first compound in a pharmaceutical composition may be less than about 5 g times 10, or about 50 grams.

In one embodiment, the amount of a first compound in a pharmaceutical composition may be at least about 0.7% of food consumed by the subject. In other embodiments, the amount of a first compound in a pharmaceutical composition may be at least about 4.3% of food consumed by the subject. In yet other embodiments, the amount of first compound in a pharmaceutical composition may be at least about 6.45% of food consumed by the subject.

Table 1 below provides experimental results that show that pharmaceutical compositions containing first compounds provide methionine restriction benefits. The left column of Table 1 below illustrates body conditions or states for male and female subjects, including total body mass, white adipose tissue mass in the form of inguinal and perigonadal fat mass, plasma glucose amounts, plasma insulin amounts, plasma IGF-1 amounts, plasma leptin amounts, plasma adiponectin amounts, and plasma FGF-21 amounts. The untreated column shows that these conditions or states are at 100% when the subjects are untreated. The methionine restriction (MR) column shows percentages of the body conditions or states for male and female subjects whose methionine intake has been restricted. These results show dietary methionine restriction benefits in the form of decreased body mass (e.g., 100% untreated versus 81% for dietary methionine restriction and 100% untreated versus 65% for dietary methionine restriction for male and female subjects respectively), decreased inguinal fat mass (100% untreated versus 52% MR and 100% untreated versus 30% MR for male and female subjects respectively), decreased perigonadal fat mass (100% untreated versus 36% MR and 100% untreated versus 24% MR for male and female subjects respectively), decreased plasma glucose amounts (100% untreated versus 69% MR and 100% untreated versus 89% MR for male and female subjects respectively), decreased plasma insulin amounts (100% untreated versus 25% MR and 100% untreated versus 28% MR for male and female subjects respectively), decreased plasma IGF-1 amounts (100% versus 59% MR and 100% versus 56% MR for male and female subjects respectively), decreased plasma leptin amounts (100% versus 15% MR and 100% versus 14% MR for male and female subjects respectively), increased plasma adiponectin amounts (100% versus 119% MR and 100% versus 147% MR for male and female subjects respectively), and increased plasma FGF-21 amounts (100% versus 3042% MR and 100% versus 846% MR for male and female subjects respectively). The remaining columns in Table 1 below show that various “first compounds” provide varying degrees of the listed benefits associated with dietary methionine restriction.

TABLE 1

| | | | | | +O-Benzyl- | +S-Phenyl- | |
|---|---|---|---|---|---|---|---|
| | | MR | Untreated | +Lysine | Serine | Cysteine | +Tryptophan |
| Total Body | Males | 81% | 100% | 91% | 89% | 87% | 85% |
| Mass | Females | 65% | 100% | 72% | 69% | 71% | — |
| Inguinal Fat | Males | 52% | 100% | 42% | 42% | 44% | 50% |
| Mass | Females | 30% | 100% | 38% | 27% | 23% | — |
| Perigonadal | Males | 36% | 100% | 34% | 31% | 35% | 39% |
| Fat Mass | Females | 24% | 100% | 37% | 25% | 25% | — |
| Plasma | Males | 69% | 100% | 84% | 93% | 70% | 94% |
| Glucose | Females | 89% | 100% | 73% | 71% | 71% | — |
| Plasma Insulin | Males | 25% | 100% | 22% | 28% | 25% | 56% |
| | Females | 28% | 100% | 41% | 37% | 32% | — |
| Plasma IGF-1 | Males | 59% | 100% | 68% | 72% | 66% | 73% |
| | Females | 56% | 100% | 66% | 79% | 72% | — |
| Plasma Leptin | Males | 14% | 100% | 13% | 12% | 11% | 18% |
| | Females | 15% | 100% | 28% | 20% | 16% | — |
| Plasma | Males | 119% | 100% | 92% | 108% | 213% | 118% |
| Adiponectin | Females | 147% | 100% | 125% | 106% | 100% | — |
| Plasma FGF-21 | Males | 3042% | 100% | — | — | — | 502% |
| | Females | 846% | 100% | 144% | 280% | 181% | — |

It will be noted that first compounds, including lysine, O-Benzyl-Serine, S-Phenyl Cysteine, and tryptophan, create a reduction or increase in a subject like methionine-restricted diets do. Accordingly, pharmaceutical compounds containing first compounds provide the methionine-restricted benefits shown in the table.

Selenium Pharmaceutical Compositions

The benefits of dietary methionine restriction may be obtained without dietary restriction by intervention or treatment with a pharmaceutical composition that, in one embodiment, includes selenium.

The selenium may include organic selenium and/or an inorganic selenium in various forms or compounds. In embodiments where the pharmaceutical composition includes organic selenium, the organic selenium compound may include at least one of selenomethionine, purified selenomethionine, 1,4-phenylenebis(methylene)selenocyanate, selenohomocysteine, selenocystathionine, selenocysteine/selenocystine, selenoglutathione trisulfide, methaneselenol, dimethyl selenide, trimethyl selenide, selenized yeast extract, and combinations thereof.

Embodiments of the pharmaceutical composition that include inorganic selenium may include pharmaceutically-acceptable salts of a selenium-based acid. By way of non-limiting example, the selenium may include selenate and/or selenite salts. In one embodiment, the selenium includes sodium selenite. In other embodiments, the selenium includes sodium selenate. Other selenium salts may also be used, including but not limited to sodium selenite, sodium selenate, cobalt selenite, cobalt selenate, selenic acid, selenious acid, selenium bromide, selenium chloride, selenium hexafluoride, selenium oxide, selenium oxybromide, selenium oxychloride, selenium oxyfluoride, selenium sulfides, selenium tetrabromide, selenium tetrachloride and selenium tetrafluoride. In other embodiments, other inorganic selenium sources may be used, such as those listed in the Merck index.

The referenced selenium compounds, either alone or in combination with each other, or in combination with other compounds described herein, may affect certain metabolic pathways within a subject to achieve a methionine-restricted state and the accompanying benefits thereof, without dietarily restricting the methionine consumption of the subject receiving the pharmaceutical composition.

The pharmaceutical composition may include an amount of selenium compound that is at least 1/6000 of the amount of sulfur amino acids consumed in the subject's diet and/or the recommended daily allowance of sulfur amino acids for the subject. In another embodiment, the pharmaceutical composition may include an amount of selenium compound that is at least about 1/1000 of the amount sulfur amino acids consumed in the subject's diet and/or the recommended daily allowance of sulfur amino acids for the subject. In another embodiment, the pharmaceutical composition may include an amount of selenium that is at least about 1/200 of the amount sulfur amino acids consumed in the subject's diet and/or the recommended daily allowance of sulfur amino acids for the subject. In another embodiment, the pharmaceutical composition may include an amount of selenium that is at least about 1/100 of the amount sulfur amino acids consumed in the subject's diet and/or the recommended daily allowance of sulfur amino acids for the subject.

In one embodiment, the daily dose amount of a selenium compound in a pharmaceutical composition administered to a subject may be about 2.4 g (the average American daily diet intake of sulfur amino acids) times 1/6000, or about 0.4 milligrams. In another embodiment, the daily dose amount of a selenium compound in a pharmaceutical composition administered to a subject may be about 2.4 g times 1/1000, or about 2.4 milligrams. In another embodiment, the daily amount of a selenium compound in a pharmaceutical composition administered to a subject may be about 2.4 g times 1/200, or about 12 milligrams. In another embodiment, the daily dose amount of selenium compound in a pharmaceutical composition administered to a subject may be about 2.4 g times 1/100, or about 24 milligrams.

In one embodiment, the pharmaceutical composition may include an amount of selenium compound that is greater than about 0.5 g (the low end of Sulfur amino acid intake range in an average diet) times 1/6000, or about 0.0833 milligrams. In another embodiment, the pharmaceutical composition may include an amount of selenium compound that is greater than about 0.5 g times 1/1000, or about 5 milligrams. In yet another embodiment the pharmaceutical composition may include an amount of selenium compound that is less than about 5 g (the high end of Sulfur amino acid intake range in an average diet) times 1/200, or about 25 milligrams. In yet another embodiment the pharmaceutical composition may include an amount of selenium compound that is less than about 5 g times 1/100, or about 50 milligrams.

In one embodiment, the effective amount of selenium compound in the pharmaceutical composition may be a percentage of total food consumed. Studies vary on the amount of food consumed by an average adult. Some studies show that the average adult consumes at least about 0.9 kg of food daily. Other studies show that the average American adult consumes about 906 kg of food annually, or about 2.5 kg daily. The pharmaceutical composition may include an amount of selenium compound that may be at least about 0.00015% of total food consumed. In other embodiments, the pharmaceutical composition may include an amount of selenium compound that may be at least 0.0036% of total food consumed. In other embodiments, the pharmaceutical composition may include an amount of selenium compound that may be at least about 0.0073% of total food consumed. Thus, in certain embodiments, multiplying the percentages of selenium of total food consumed by the various average adult consumptions of daily food consumed, the daily dose of selenium compound may be at least about 1.35 mg, at least about 32.4 mg, or at least about 65.7 mg. In other embodiments, the amount of selenium compound may be about 3.75 mg, about 90.0 mg, or about 182.5 mg. Experimental data presented herein and described below indicates that the extent of methionine restriction-like benefits produced by administration with various amounts of selenium compounds is dose-dependent. For example, the benefits resulting from a selenium dosage of 0.00015% of total food consumed are lesser in magnitude than those resulting from a selenium dosage of 0.0036%, which in turn, are lesser in magnitude than those resulting from a selenium dosage of 0.0073%. Accordingly, the data suggests that a percentage of selenium, such as 0.000015% of total food consumed, or in other words, a percentage that is just higher than what might be attributed to experimental variance, would be enough to provide a methionine restriction benefit.

Table 2 below shows that pharmaceutical compositions containing selenium compounds provide methionine restriction benefits. As with Table 1 above, the left column of Table 2 below illustrates body conditions or states for male and female subjects, including total body mass, white adipose tissue mass in the form of inguinal and perigonadal fat mass, plasma glucose amounts, plasma insulin amounts, plasma IGF-1 amounts, plasma leptin amounts, plasma adiponectin amounts, and plasma FGF-21 amounts. The untreated column shows that these conditions or states as 100% when the subjects are untreated. The methionine restriction (MR) column shows percentages of the body conditions or states for male and female subjects whose methionine intake has been restricted. Similar to Table 1, Table 2 results show the dietary methionine restriction benefits of decreased body mass, decreased inguinal fat mass, decreased perigonadal fat mass, decreased plasma glucose amounts decreased plasma insulin amounts, decreased plasma IGF-1 amounts, decreased plasma leptin amounts, increased plasma adiponectin amounts, and increased plasma FGF-21 amounts. The remaining columns in Table 2 below show that organic and inorganic selenium compounds provide varying degrees of the benefits associated with dietary methionine restriction.

TABLE 2

| | | Untreated | +Sodium Selenite | +Selenomethionine | MR |
|---|---|---|---|---|---|
| Total Body Mass | Males | 100% | 60% | 84% | 60% |
| | Females | 100% | 66% | 79% | 74% |
| Inguinal Fat Mass | Males | 100% | 17% | 54% | 17% |
| | Females | 100% | 19% | 48% | 47% |
| Perigonadal Fat Mass | Males | 100% | 20% | 60% | 22% |
| | Females | 100% | 20% | 55% | 44% |
| Plasma Glucose | Males | 100% | 71% | — | 56% |
| | Females | 100% | 69% | — | 84% |
| Plasma Insulin | Males | 100% | 19% | — | 6% |
| | Females | 100% | 25% | — | 29% |
| Plasma IGF-1 | Males | 100% | 63% | — | 47% |
| | Females | 100% | 35% | — | 67% |
| Plasma Leptin | Males | 100% | 4% | — | 2% |
| | Females | 100% | 17% | — | 34% |
| Plasma Adiponectin | Males | 100% | 128% | — | 201% |
| | Females | 100% | 134% | — | 130% |
| Plasma FGF-21 | Males | 100% | 3045% | — | 1382% |
| | Females | 100% | 3971% | — | 1707% |

It will be noted that selenium compounds, including organic selenium (such as selenomethionine by way of non-limiting example) and inorganic selenium (such as sodium selenite, by way of non-limiting example) create a reduction or increase in a subject like methionine-restricted diets do. Accordingly, pharmaceutical compounds containing selenium compounds provide the methionine restriction benefits shown in the table. Thus, as can be seen by reference to Tables and 1 and 2, the pharmaceutical composition of the present invention, includes at least one of a first compound and a selenium compound in an amount such that administration of the pharmaceutical composition to a subject, causes the subject to experience one or more of reduced body mass, reduced inguinal fat mass, reduced perigonadal fat mass, reduced plasma glucose, reduced plasma insulin, reduced plasma IGF-1, reduced plasma leptin, increased plasma adiponectin and increased plasma FGF-21, as compared to a subject not treated with the pharmaceutical composition.

Formation of Pharmaceutical Compositions

The pharmaceutical composition of the invention can be created through various methods known in the art such as dry granulation, wet granulation, melt granulation, direct compression, double compression, extrusion spherization, layering, conventional mixing, dissolution, dragee, crushing, emulsification, encapsulation, entrapment or lyophilization processes, and the like. The solvent or solvents used in wet granulation formation embodiments include all the solvents well known in the art or their mixtures thereof. In one embodiment the pharmaceutical composition and/or individual compounds contained therein may be partially or completely dehydrated. In other embodiments, the pharmaceutical composition and/or components thereof may be mono hydrated. In other embodiments, the pharmaceutical composition and/or its constituents may be in powder form. In yet another embodiment, the pharmaceutical composition may be water soluble.

The pharmaceutical compositions described herein may include certain additional pharmaceutically-acceptable materials or substances such as, without limitation, carriers, excipients, stabilizers, buffers, lubricants, time-release agents, wetting agents, emulsifiers, pressure regulating substances, and/or coatings. In certain embodiments, additional pharmaceutically-acceptable materials or substances may also include adjuvants, antipruritic, astringent, local anesthetics and/or anti-inflammatory agents. Additional pharmaceutically-acceptable materials or substances may be materials or substances that would be useful in creating various dosage forms of the pharmaceutical compositions. In yet other embodiments, pharmaceutically-acceptable materials or substances may include colorants, flavoring agents, preservatives, antioxidants, opacifiers, and/or thickening agents.

Additional pharmaceutically-acceptable materials or substances may assist in the administration of the pharmaceutical composition, the facilitation of the processing of the pharmaceutical compositions in preparations that can be used pharmaceutically, and/or the effectiveness of the pharmaceutical composition. These pharmaceutically-acceptable materials or substances may perform one or more functions. For examples of carriers, stabilizers, and adjuvants, see, e.g., Martin, Remington's Pharmaceutical Sciences, 15th Ed., Mack Publ. Co., Easton, Pa. (1975), relevant portions incorporated herein by reference. For examples of excipients, see, e.g., the Handbook of Pharmaceutical Excipients, American Pharmaceutical Association (1986), relevant portions incorporated by reference herein.

It will be appreciated by those of skill in the art that any such pharmaceutically-acceptable materials or substances, when added, should not unduly interfere with the biological activities of the components of the pharmaceutical compositions of the present invention. It will further be appreciated that that the amount or dosage of pharmaceutical composition and any additional pharmaceutically-acceptable materials or substances may depend on many factors, including subject height, weight, body surface area, age, gender, the subject's general state of health, other subject characteristics, the specific compound to be administered, the time of administration, the route of administration, any interaction with other drugs that are being administered simultaneously, and the like.

The amount and kind of pharmaceutically-acceptable materials or substances may further depend upon how much pharmaceutical composition is used and/or how the pharmaceutical composition is to be used. For example, in one embodiment, the pharmaceutical composition includes pharmaceutically-acceptable materials or substances that form a delivery system to target an area of the gut or digestive tract for release of the pharmaceutical composition within the subject. These target areas may include the stomach, large intestine, the small intestine, duodenum, other areas of the gastrointestinal tract, intercellular locations, and the like. Accordingly, in certain embodiments, the delivery system may include a time release agent, a coating, similar or varying core densities, soluble and insoluble materials, and the like. Extended or controlled-release formulation embodiments of the present invention may include one or more excipients that slow the release of the agents by coating or temporarily bonding or decreasing the solubility of the pharmaceutical composition or components thereof. Examples of such excipients may include cellulose ethers such as hydroxypropyl methylcellulose, polyvinylacetate-based excipients, and polymers and copolymers based on methacrylates and methacrylic acid. These additional pharmaceutically-acceptable materials may be positioned or formed in or around individual and/or specific compounds or constituents of the pharmaceutical compositions to effect the desired delivery.

After, or as part of, the creation of the pharmaceutical compositions, including any pharmaceutically-acceptable material or substances used, the pharmaceutical compositions may be put into dosage forms for administration. The dosage form, along with any auxiliary materials necessary or desired to create the dosage form, may also depend on the type of administration. In some embodiments, administrative routes for the pharmaceutical compositions described herein may include, for example, oral or transmucosal administration, as well as parenteral release, including intramuscular, subcutaneous, intramedullary, intrathecal, intraventricular, intravenous, intraperitoneal, intramuscularly, intrapulmonary, vaginally, rectally, or intranasal administration, and the like. Accordingly, dosage form embodiments of the present invention may be configured to accommodate these administration routes. In some embodiments, the pharmaceutical compositions of the present invention may include any number of dosage forms and/or delivery vehicles, including without limitation, ingestibles, digestibles, injectables, I.V. drips, topicals, inhalants, nasal sprays, patches, absorbing gels, salves, lotions, creams, liquids, liquid tannates, suppositories, enemas, and the like, including without limitation, tablets, pills, capsules, aqueous and non-aqueous liquids, gels, syrups, suspensions, mixed-media, and the like.

In some embodiments, the pharmaceutical composition, with any additional matter or substances, may be in a solid dosage form, including without limitation, discrete units of capsules, pills, or tablets, or as a powder or granule. The solid dosage form may be formed by compression molding. Such compressed tablets may be prepared by compressing pharmaceutical compositions and any additional pharmaceutically-acceptable substance or material in a suitable machine. The molded tablets can be optionally coated, scored, or otherwise treated or prepared to effectuate a controlled release of the pharmaceutical composition or one or more of the first or selenium compounds. Where the pharmaceutical composition is in pill form, the size of the punch, and/or the resulting size of the pill may be configured for retaining and/or releasing the pharmaceutical composition in a predetermined or particular part of the gut. By way of non-limiting example, a bigger punch may provide a larger pill, which may keep the pharmaceutical composition from passing out of the upper part of the stomach. Accordingly, the pharmaceutical composition may, in one embodiment, be formed with an average size or other characteristic of a subject's anatomy in mind. By way of non-limited example, where the subject's gastric sphincter is about 12 mm, the pharmaceutical composition may be formed so that its diameter is about 13 mm.

In some embodiments, the pharmaceutical compositions may be configured for parenteral administration and may include aqueous solutions, such as Hank's solution, Ringer's solution, or a physiologically buffered saline. In other embodiments, the pharmaceutical composition, with any additional materials or substances, may be in a form suitable for tissue or cellular administration and may include suitable penetrants, known in the art to cross tissue or cellular barriers. Additional embodiments of the pharmaceutical compositions may include suitable oily suspensions for injection. Other embodiments may include suitable lipophilic solvents or vehicles and may include fatty oils such as sesame oil, or esters of synthetic fatty acids, such as ethyl oleate or triglycerides, or liposomes. Aqueous injectable suspensions may contain substances that increase the viscosity of the suspension, such as sodium carboxymethyl cellulose, sorbitol, or dextran.

In some embodiments the pharmaceutical composition may be administered topically to the skin of a subject. The pharmaceutical composition may be mixed with a pharmaceutically-acceptable carrier or a base which is suitable for topical application to skin to form a dermatological composition. Suitable examples of carrier or base include, but not limited to, water, glycols, alcohols, lotions, creams, gels, emulsions, and sprays. The pharmaceutical composition with the carrier or base may be integrated into a topical dressing, medicated tape, dermal patch, absorbing gel, and other administrative vehicles.

Formulation techniques for the embodiments of pharmaceutical compositions of the present invention can be found in the latest edition of Remington's Pharmaceutical Sciences (Ed. Mack Publishing Co, Easton, Pa.). Pharmaceutical compositions suitable for use in the present invention include compositions or compounds which in an amount effective to provide a methionine restriction benefit without dietarily restricting methionine consumption, the benefits including without limitation, IGF-1 down regulation and protection against obesity-related diseases and disorders.

It should be noted that for pharmaceutical compositions containing selenium, the FDA has established selenium nutritional levels with recommend amounts for humans that can be safely metabolized. Although limited amounts of both organic and inorganic forms of selenium may provide the benefits of the novel composition and method embodiments described herein, chronic administration of high concentrations of selenium can be toxic. In the composition, method and use embodiments described herein, an effective amount of a pharmaceutical composition containing selenium is less than an amount that would be predicted to be toxic to humans or animals.

### Methods

Pharmaceutical compositions described herein may be used in treatment methods to enhance lifespan.

Providing a Methionine Restriction Benefit

In one embodiment, a method for providing a benefit associated with a methionine-restricted diet to a subject without restricting the subject's methionine consumption includes administering a pharmaceutical composition to the subject that includes an effective amount of one or more of a first compound and a selenium compound. The pharmaceutical composition and/or the constituents thereof may be any of those described herein. Accordingly, the method may include administering a pharmaceutical composition that includes at least one first compound chosen from lysine, O-benzyl serine (OBS), S-phenyl cysteine (SPC), S-benzyl cysteine (SBC), isoleucine, taurine, tryptophan, and 2-aminobicyclo-(2.2.1)-heptane-2-carboxylic acid (BCH). These first compounds may be used alone or in combination with each other and/or other compounds.

The method may also include administering a pharmaceutical composition that includes selenium. The selenium may be an organic or inorganic compound. Where the pharmaceutical composition includes organic selenium, the selenium compounds may include at least one of selenomethionine, purified selenomethionine, 1,4-phenylenebis(methylene)selenocyanate, selenohomocysteine, selenocystathionine, selenocysteine/selenocystine, selenoglutathione trisulfide, methaneselenol, dimethyl selenide, trimethyl selenide, and selenized yeast extract. These organoselenium compounds may be used alone or in combination with each other and/or other compounds. Where the pharmaceutical composition includes inorganic selenium, the selenium may include a pharmaceutically-acceptable salt of a selenium-based acid. In one embodiment, the inorganic selenium includes sodium selenite.

The method may provide any of the methionine restriction benefits described herein. These may include without limitation, at least one benefit chosen from reduced adipose tissue accumulation, reduced body mass, reduced fatty liver, reduced circulating levels of IGF-1, increased circulating levels of FGF-21, reduced circulating levels of leptin, increased circulating levels of adiponectin, reduced circulating levels of glucose, and reduced circulating levels of insulin.

In one embodiment, the method of providing a methionine restriction benefit includes administering a pharmaceutical composition that includes an effective amount of a first compound. The effective amount of first compound may be any of the amounts of first compound described herein in connection with a pharmaceutical composition. In certain embodiments, the effective amount of a first compound in a pharmaceutical composition may be determined in relation to the subject's typical sulfur amino acid intake. In other embodiments, the effective amount of a first compound may be determined in relation to a recommended daily allowance of sulfur amino acid for the subject. Accordingly, administering an effective amount of a first compound may include administering a pharmaceutical composition containing a first compound that is at least about 2.5 times the amount of sulfur amino acids in the subject's diet. In other embodiments, administering an effective amount of a first compound may include administering a pharmaceutical composition containing a first compound that is at least about 5 times the amount of sulfur amino acids in the subject's diet. In other embodiments, the effective amount first compound is less than about 10 times the amount of sulfur amino acids in the subject's diet. In yet other embodiments, the effective amount of first compound in a pharmaceutical composition to be administered is less than about 7.5 times the amount of sulfur amino acids in the subject's diet.

An effective amount of pharmaceutical composition or constituents thereof to be administered to a subject may be determined by weight. In one embodiment, the effective amount of first compound to be administered by way of a pharmaceutical composition to a subject in order to provide a methionine restriction benefit is at least about 1.25 g. In other embodiments, the amount may be less than about 50 g. An affective amount may also be any of the amounts defined by weight described herein throughout. An effective amount of pharmaceutical composition or constituents thereof to be administered to a subject may also be determined as a percentage of the subject food consumption. In one embodiment, the effective amount of a first compound in a pharmaceutical composition includes at least about 4.3% of food consumed by the subject. In other embodiments, the effective amount of a first compound includes at least about 6.45% of food consumed by the subject. The effective amount of first compound in a pharmaceutical composition may be an amount that causes the treated subject to achieve the results set forth in Table 1 and the Figures attached hereto.

As is shown in Table 1 above, an effective amount of a first compound in a pharmaceutical composition may be an amount that causes the treated subject to gain at least about 9% less body mass as compared to an untreated subject. In other embodiments, an effective amount of first compound in a pharmaceutical composition may be an amount that causes the treated subject to gain at least about 11% less body mass as compared to an untreated subject. In other embodiments, an effective amount of first compound in a pharmaceutical composition may be an amount that causes the treated subject to gain at least about 13% less body mass as compared to an untreated subject. In other embodiments, a first compound in a pharmaceutical composition may be an amount that causes the treated subject to gain at least about 15% less body mass as compared to an untreated subject. In other embodiments, an effective amount of first compound in a pharmaceutical composition may be an amount that causes the treated subject to gain at least about 28% less body mass as compared to an untreated subject. In other embodiments, an effective amount of first compound in a pharmaceutical composition may be an amount that causes the treated subject to gain at least about 29% less body mass as compared to an untreated subject. In other embodiments, an effective amount of first compound in a pharmaceutical composition may be an amount that causes the treated subject to gain at least about 31% less body mass as compared to an untreated subject.

As is further shown in Table 1 above, an effective amount of first compound in a pharmaceutical composition may be an amount that causes the treated subject to accumulate at least about 50% less white adipose tissue as compared to an untreated subject. Indeed, an effective amount of first compound in a pharmaceutical composition may be an amount that causes the treated subject to accumulate at least about 50% less inguinal fat mass as compared to an untreated subject. In other embodiments, an effective amount of first compound in a pharmaceutical composition may be an amount that causes the treated subject to accumulate at least about 56% less inguinal fat mass as compared to an untreated subject. In other embodiments, an effective amount of first compound in a pharmaceutical composition may be an amount that causes the treated subject to accumulate at least about 58% less inguinal fat mass as compared to an untreated subject. In other embodiments, an effective amount of first compound in a pharmaceutical composition may be an amount that causes the treated subject to accumulate at least about 62% less inguinal fat mass as compared to an untreated subject. In other embodiments, an effective amount of first compound in a pharmaceutical composition may be an amount that causes the treated subject to accumulate at least about 73% less inguinal fat mass as compared to an untreated subject. In other embodiments, an effective amount of first compound in a pharmaceutical composition may be an amount that causes the treated subject to accumulate at least about 77% less inguinal fat mass as compared to an untreated subject.

As is further shown in Table 1 above, an effective amount of first compound in a pharmaceutical composition may be an amount that causes the treated subject to accumulate at least about 61% less white adipose tissue as compared to untreated subjects. Specifically, an effective amount of first compound in a pharmaceutical composition may be an amount that causes the treated subject to accumulate at least about 61% less perigonadal fat mass as compared to an untreated subject. In other embodiments, an effective amount of first compound in a pharmaceutical composition may be an amount that causes the treated subject to accumulate at least about 63% less perigonadal fat mass as compared to an untreated subject. In other embodiments, an effective amount of first compound in a pharmaceutical composition may be an amount that causes the treated subject to accumulate at least about 65% less perigonadal fat mass as compared to an untreated subject. In other embodiments, an effective amount of first compound in a pharmaceutical composition may be an amount that causes the treated subject to accumulate at least about 66% less perigonadal fat mass as compared to an untreated subject. In other embodiments, an effective amount of first compound in a pharmaceutical composition may be an amount that causes the treated subject to accumulate at least about 69% less perigonadal fat mass as compared to an untreated subject. In other embodiments, an effective amount of first compound in a pharmaceutical composition may be an amount that causes the treated subject to accumulate at least about 75% less perigonadal fat mass as compared to an untreated subject.

As is further shown in Table 1 above, an effective amount of first compound in a pharmaceutical composition may be an amount that causes the treated subject to accumulate at least about 6% less plasma glucose as compared to an untreated subject. In other embodiments, an effective amount of first compound in a pharmaceutical composition may be an amount that causes the treated subject to accumulate at least about 7% less plasma glucose as compared to an untreated subject. In other embodiments, an effective amount of first compound in a pharmaceutical composition may be an amount that causes the treated subject to accumulate at least about 16% less plasma glucose as compared to an untreated subject. In other embodiments, an effective amount of first compound in a pharmaceutical composition may be an amount that causes the treated subject to accumulate at least about 27% less plasma glucose as compared to an untreated subject. In other embodiments, an effective amount of first compound in a pharmaceutical composition may be an amount that causes the treated subject to accumulate at least about 29% less plasma glucose as compared to an untreated subject. In other embodiments, an effective amount of first compound in a pharmaceutical composition may be an amount that causes the treated subject to accumulate at least about 30% less plasma glucose as compared to an untreated subject.

As is further shown in Table 1 above, an effective amount of first compound in a pharmaceutical composition may be an amount that causes the treated subject to accumulate at least about 44% less plasma insulin as compared to an untreated subject. In other embodiments, an effective amount of first compound in a pharmaceutical composition may be an amount that causes the treated subject to accumulate at least about 59% less plasma insulin as compared to an untreated subject. In other embodiments, an effective amount of first compound in a pharmaceutical composition may be an amount that causes the treated subject to accumulate at least about 63% less plasma insulin as compared to an untreated subject. In other embodiments, an effective amount of first compound in a pharmaceutical composition may be an amount that causes the treated subject to accumulate at least about 68% less plasma insulin as compared to an untreated subject. In other embodiments, an effective amount of first compound in a pharmaceutical composition may be an amount that causes the treated subject to accumulate at least about 71% less plasma insulin as compared to an untreated subject. In other embodiments, an effective amount of first compound in a pharmaceutical composition may be an amount that causes the treated subject to accumulate at least about 72% less plasma insulin as compared to an untreated subject. In other embodiments, an effective amount of first compound in a pharmaceutical composition may be an amount that causes the treated subject to accumulate at least about 75% less plasma insulin as compared to an untreated subject.

As is further shown in Table 1 above, an effective amount of first compound in a pharmaceutical composition may be an amount that causes the treated subject to accumulate at least about 21% less plasma IGF-1 as compared to an untreated subject. In other embodiments, an effective amount of first compound in a pharmaceutical composition may be an amount that causes the treated subject to accumulate at least about 27% less plasma IGF-1 as compared to an untreated subject. In other embodiments, an effective amount of first compound in a pharmaceutical composition may be an amount that causes the treated subject to accumulate at least about 28% less plasma IGF-1 as compared to an untreated subject. In other embodiments, an effective amount of first compound in a pharmaceutical composition may be an amount that causes the treated subject to accumulate at least about 32% less plasma IGF-1 as compared to an untreated subject. In other embodiments, an effective amount of first compound in a pharmaceutical composition may be an amount that causes the treated subject to accumulate at least about 34% less plasma IGF-1 as compared to an untreated subject.

As is further shown in Table 1 above, an effective amount of first compound in a pharmaceutical composition may be an amount that causes the treated subject to accumulate at least about 72% less plasma leptin as compared to an untreated subject. In other embodiments, an effective amount of first compound in a pharmaceutical composition may be an amount that causes the treated subject to accumulate at least about 80% less plasma leptin as compared to an untreated subject. In other embodiments, an effective amount of first compound in a pharmaceutical composition may be an amount that causes the treated subject to accumulate at least about 82% less plasma leptin as compared to an untreated subject. In other embodiments, an effective amount of first compound in a pharmaceutical composition may be an amount that causes the treated subject to accumulate at least about 84% less plasma leptin as compared to an untreated subject. In other embodiments, an effective amount of first compound in a pharmaceutical composition may be an amount that causes the treated subject to accumulate at least about 87% less plasma leptin as compared to an untreated subject. In other embodiments, an effective amount of first compound in a pharmaceutical composition may be an amount that causes the treated subject to accumulate at least about 88% less plasma leptin as compared to an untreated subject. In other embodiments, an effective amount of first compound in a pharmaceutical composition may be an amount that causes the treated subject to accumulate at least about 89% less plasma leptin as compared to an untreated subject.

As is further shown in Table 1 above, an effective amount of first compound in a pharmaceutical composition may be an amount that causes the treated subject to accumulate at least about 6% more plasma adiponectin as compared to an untreated subject. In other embodiments, an effective amount of first compound in a pharmaceutical composition may be an amount that causes the treated subject to accumulate at least about 8% more plasma adiponectin as compared to an untreated subject. In other embodiments, an effective amount of first compound in a pharmaceutical composition may be an amount that causes the treated subject to accumulate at least about 18% more plasma adiponectin as compared to an untreated subject. In other embodiments, an effective amount of first compound in a pharmaceutical composition may be an amount that causes the treated subject to accumulate at least about 25% more plasma adiponectin as compared to an untreated subject. In other embodiments, an effective amount of first compound in a pharmaceutical composition may be an amount that causes the treated subject to accumulate at least about 113% more plasma adiponectin as compared to an untreated subject.

As is further shown in Table 1 above, an effective amount of first compound in a pharmaceutical composition may be an amount that causes the treated subject to accumulate at least about 44% more plasma FGF-21 as compared to an untreated subject. In other embodiments, an effective amount of first compound in a pharmaceutical composition may be an amount that causes the treated subject to accumulate at least about 81% more plasma FGF-21 as compared to an untreated subject. In other embodiments, an effective amount of first compound in a pharmaceutical composition may be an amount that causes the treated subject to accumulate at least about 180% more plasma FGF-21 as compared to an untreated subject. In other embodiments, an effective amount of first compound in a pharmaceutical composition may be an amount that causes the treated subject to accumulate at least about 402% more plasma FGF-21 as compared to an untreated subject.

In one embodiment, the method of providing a methionine restriction benefit includes administering a pharmaceutical composition that includes an effective amount of a selenium compound. The effective amount of selenium compound may be any of the amounts of selenium compound described herein in connection with a pharmaceutical composition.

Where the pharmaceutical composition contains selenium, the effective amount of selenium to be administered to a subject in order to provide a methionine restriction benefit may likewise be determined in relation to the subject's sulfur amino acid levels, the subject's food consumption, the weight of the pharmaceutical composition, and/or the desired results to be achieved. The administration of an effective amount of selenium in pharmaceutical composition to be administered to a subject in order to provide a methionine restriction benefit includes any of the effective amounts described herein in connection with selenium.

Administering an effective amount of pharmaceutical composition containing selenium may include administering an effective amount of selenium that is at least about 1/6000 times the amount of sulfur amino acids by weight in the subject's diet. In other embodiments, the effective amount of selenium is at least about 1/1000 times the amount of sulfur amino acids in the subject's diet. In other embodiments, the effective amount of selenium is at least about 1/200 times the amount of sulfur amino acids in the subject's diet. In yet other embodiments, the effective amount of selenium is at least about 1/100 times the amount of sulfur amino acids in the subject's diet.

In one embodiment, the effective amount of selenium compound in a pharmaceutical composition to be administered to a subject in order to provide a methionine restriction benefit is at least about 0.0833 mg. In other embodiments, the effective amount may be less than about 182.5 mg. In one embodiment, the effective amount of selenium in the pharmaceutical composition includes at least about 0.00015% of food consumed by the subject. In other embodiments, the effective amount of a selenium compound includes at least about 0.0036% of food consumed by the subject. In other embodiments, the effective amount of a selenium compound includes at least about 0.0073% of food consumed by the subject.

The effective amount of a selenium compound may be an amount that causes the subject treated with a pharmaceutical composition containing the selenium compound to achieve the results set forth in Table 2 and the figures attached hereto.

As shown in Table 2 above, an effective amount of selenium compound in a pharmaceutical composition may be an amount that causes the treated subject to gain at least about 16% less body mass as compared to an untreated subject. In other embodiments, an effective amount of selenium compound in a pharmaceutical composition may be an amount that causes the treated subject to gain at least about 21% less body mass as compared to an untreated subject. In other embodiments, an effective amount of selenium compound in a pharmaceutical composition may be an amount that causes the treated subject to gain at least about 34% less body mass as compared to an untreated subject. In other embodiments, an effective amount of selenium compound in a pharmaceutical composition may be an amount that causes the treated subject to gain at least about 40% less body mass as compared to an untreated subject.

As further shown in Table 2 above, an effective amount of selenium compound in a pharmaceutical composition may be an amount that causes the treated subject to accumulate at least about 40% less white adipose tissue as compared to an untreated subject. Indeed, an effective amount of selenium compound in a pharmaceutical composition may be an amount that causes the treated subject to accumulate at least about 40% less inguinal fat mass as compared to an untreated subject. In other embodiments, an effective amount of selenium compound in a pharmaceutical composition may be an amount that causes the treated subject to accumulate at least about 52% less inguinal fat mass as compared to an untreated subject. In other embodiments, an effective amount of selenium compound in a pharmaceutical composition may be an amount that causes the treated subject to accumulate at least about 58% less inguinal fat mass as compared to an untreated subject. In other embodiments, an effective amount of selenium compound in a pharmaceutical composition may be an amount that causes the treated subject to accumulate at least about 81% less inguinal fat mass as compared to an untreated subject. In other embodiments, an effective amount of selenium compound in a pharmaceutical composition may be an amount that causes the treated subject to accumulate at least about 83% less inguinal fat mass as compared to an untreated subject.

As further shown in Table 2 above, an effective amount of selenium compound in a pharmaceutical composition may be an amount that causes the treated subject to accumulate at least about 40% less white adipose tissue in the form of perigonadal fat mass as compared to an untreated subject. In other embodiments, an effective amount of selenium compound in a pharmaceutical composition may be an amount that causes the treated subject to accumulate at least about 45% less perigonadal fat mass as compared to an untreated subject. In other embodiments, an effective amount of selenium compound in a pharmaceutical composition may be an amount that causes the treated subject to accumulate at least about 80% less perigonadal fat mass as compared to an untreated subject.

As further shown in Table 2 above, an effective amount of a pharmaceutical composition containing an inorganic selenium compound may be an amount that causes the treated subject to accumulate at least about 29% less plasma glucose as compared to an untreated subject. In other embodiments, an effective amount of a pharmaceutical composition containing an inorganic selenium compound may be an amount that causes the treated subject to accumulate at least about 31% less plasma glucose as compared to an untreated subject.

As further shown in Table 2 above, an effective amount of an inorganic selenium compound in a pharmaceutical composition may be an amount that causes the treated subject to accumulate at least about 75% less plasma insulin as compared to an untreated subject. In other embodiments, an effective amount of an inorganic selenium compound may be an amount that causes the treated subject to accumulate at least about 81% less plasma insulin as compared to an untreated subject.

As further shown in Table 2 above, an effective amount of an inorganic selenium compound in a pharmaceutical composition may be an amount that causes the treated subject to accumulate at least about 37% less plasma IGF-1 as compared to an untreated subject. In other embodiments, an effective amount of selenium compound in a pharmaceutical composition may be an amount that causes the treated subject to accumulate at least about 65% less plasma IGF-1 as compared to an untreated subject.

As further shown in Table 2 above, an effective amount of an inorganic selenium compound in a pharmaceutical composition may be an amount that causes the treated subject to accumulate at least about 83% less plasma leptin as compared to an untreated subject. In other embodiments, an effective amount of an inorganic selenium compound in a pharmaceutical composition may be an amount that causes the treated subject to accumulate at least about 96% less plasma leptin as compared to an untreated subject.

As further shown in Table 2 above, an effective amount of an inorganic selenium compound in a pharmaceutical composition may be an amount that causes the treated subject to accumulate at least about 28% more plasma adiponectin as compared to an untreated subject. In other embodiments, an effective amount of an inorganic selenium compound in a pharmaceutical composition may be an amount that causes the treated subject to accumulate at least about 34% more plasma adiponectin as compared to an untreated subject.

As further shown in Table 2 above, an effective amount of an inorganic selenium compound in a pharmaceutical composition may be an amount that causes the treated subject to accumulate at least about 2945% more plasma FGF-21 as compared to an untreated subject. In other embodiments, an effective amount of an inorganic selenium compound may be an amount that causes the treated subject to accumulate at least about 3871% more plasma FGF-21 as compared to an untreated subject.

Downregulating IGF-1 Signaling

Embodiments of the present invention describe a novel approach to achieve health span benefits similar to those of methionine restriction by decreasing IGF-1 signaling in a subject. This may be accomplished by reducing plasma IGF-1 in the subject. In one embodiment, a method of downregulating IGF-1 signaling in a subject includes administering pharmaceutical compositions including effective amounts of at least one first or “methionine competitor” compound and/or at least one selenium compound.

One way that decreased IGF-1 signaling may occur is by administering a pharmaceutical composition that includes a first or methionine competitor compound that reduces the production and/or release of IGF-1. The first compound may affect other body mechanisms to reduce the amount of plasma IGF-1 within the subject. In one embodiment, the inhibition of methionine absorption into the blood stream reduces the production of IGF-1 in the liver and/or the release of IGF-1 from the liver into the blood stream. The first compound described herein throughout may be configured to inhibit B⁰AT1 transporter amino acids from up taking methionine. By this, or other mechanisms, decreased IGF-1 signaling may occur. As discussed above, the first compound may include without limitation, lysine, O-benzyl serine (OBS), S-phenyl cysteine (SPC), S-benzyl cysteine (SBC), isoleucine, taurine, tryptophan, and 2-aminobicyclo-(2.2.1)-heptane-2-carboxylic acid (BCH). These compounds may be used alone or in combination with each and/or other compounds, including without limitation the selenium compounds discussed below, to achieve downregulated IGF-1 signaling in the subject.

In another embodiment, a method for decreasing or downregulating IGF-1 signaling may occur by administering a pharmaceutical composition that contains selenium. The selenium may be absorbed into the subject's bloodstream to affect the subject's ability to produce and/or release the hormone IGF-1. The pharmaceutical composition used to downregulate IGF-1 signaling within the subject may include a selenium compound containing organic and/or inorganic sources of selenium and may include any of the selenium-containing pharmaceutical compositions described herein. Accordingly, in one embodiment, the selenium compound may contain at least one compound chosen from selenomethionine, 1,4-phenylenebis (methylene) selenocyanate, selenohomocysteine, selenocystathionine, selenocysteine/selenocystine, selenoglutathione trisulfide, methaneselenol, dimethyl selenide, trimethyl selenide, purified selenomethionine, selenized yeast extract, and pharmaceutically-acceptable salts of selenium-based acids. These compounds may be used alone or in combination with each other or other compounds, including without limitation the first compounds discussed above, to achieve downregulated IGF-1 signaling in a subject administered to with the pharmaceutical composition.

The pharmaceutical composition or constituents thereof used to downregulate IGF-1 in a subject may include any of the pharmaceutical compositions described herein containing effective amounts of first compounds and/or selenium compounds. These effective amounts may be in terms of weight, multiples of the subject's sulfur amino acid intake or recommended intake, percentages of food consumption, and may include amounts of first and/or selenium compounds as described herein. Effective amount of first and/or selenium compounds may also be amounts used to obtain the results shown in Tables 1, 2, and the Figures attached hereto, as described herein throughout.

Treating Obesity-Related Diseases

Compositions and methods of the present invention described herein may also be used to treat obesity-related diseases. In one embodiment, a method of treating a disease or disorder associated with diet-induced obesity, low metabolism, hyperinsulinemia, adipose tissue accumulation, fatty liver, and/or combinations thereof in a subject includes administering pharmaceutical compositions, such as those described herein. Embodiments of the pharmaceutical compositions may include a first compound that contains at least one of lysine, O-benzyl serine (OBS), S-phenyl cysteine (SPC), S-benzyl cysteine (SBC), isoleucine, taurine, tryptophan, and 2-aminobicyclo-(2.2.1)-heptane-2-carboxylic acid (BCH) as described herein. Additionally, the pharmaceutical composition may include selenium or selenium compounds as described herein. The selenium compound may contain organic and/or inorganic sources of selenium. Accordingly, in one embodiment, the selenium compound may contain at least one compound chosen from selenomethionine, 1,4-phenylenebis (methylene) selenocyanate, selenohomocysteine, selenocystathionine, selenocysteine/selenocystine, selenoglutathione trisulfide, methaneselenol, dimethyl selenide, trimethyl selenide, purified selenomethionine, selenized yeast extract, and pharmaceutically-acceptable salts of selenium-based acids. These compounds may be used alone or in combination with each other or other compounds, including without limitation the first compounds discussed above, to treat obesity-related diseases or disorders in a subject.

A pharmaceutical composition containing an effective amount of at least one of a first compound and a selenium compound for use in treating an obesity-related disease or disorder may include any of the amounts of first compound and/or selenium compound described herein. These effective amounts may be in terms of weight, multiples of the subject's sulfur amino acid intake or recommended intake, and/or percentages of food consumption as discussed above. Effective amount of these compounds for treating obesity-related diseases or disorders may include amounts used to obtain the results in Tables 1, 2, and the Figures attached hereto.

Methods of Administration

The embodiments of each and/or all of the methods described above may include systemic or local administration of effective amounts of the pharmaceutical compositions and/or pharmaceutical composition constituents described herein. Each or any of these methods may include any number of administration routes, including without limitation, orally, transmucosally, parenterally, including topically, intramuscularly, subcutaneously, intramedullarily, intrathecally, intraventricularly, intravenously, intraperitoneally, intrapulmony, vaginally, rectally, intranasally, intercellular, and the like. Each or all of the methods described above may include the step of formation into a dosage form to accommodate these administration routes. In some embodiments, methods of administering pharmaceutical compositions may include forming the pharmaceutical composition, and any additional pharmaceutically-acceptable material or substance into at least one of an ingestible, a digestible, an injectable, a tablet, a pill, a capsule, an I.V. drip, a topical, an inhalant, a nasal spray, a patch, an absorbing gel, a salve, a lotion, a cream, an aqueous liquid, a non-aqueous liquid, a liquid tannate, a suspension, a syrup, a suppository, an enema, a mixed media formulation, and the like.

Medicaments for Treating Dietary Diseases

In one embodiment, a pharmaceutical composition that includes at least one of a first compound and a selenium compound may be used in the manufacture of a medicament for providing a benefit associated with a methionine-restricted diet to a subject without restricting the subject's methionine consumption. Embodiments also include pharmaceutical compositions described herein for use in the manufacture of a medicament for use in the methods described herein, including without limitation, downregulating IGF-1 signaling and treating diseases and disorders associated with diet-induced obesity. The first compound of the pharmaceutical composition may include at least one of lysine, O-benzyl serine (OBS), S-phenyl cysteine (SPC), S-benzyl cysteine (SBC), isoleucine, taurine, tryptophan, 2-aminobicyclo-(2.2.1)-heptane-2-carboxylic acid (BCH) and/or combinations thereof. The selenium compound may include at least one compound chosen from selenomethionine, selenohomocysteine, selenocystathionine, selenocysteine/selenocystine, selenoglutathione trisulfide, methaneselenol, dimethyl selenide, trimethyl selenide, purified selenomethionine, selenized yeast extract, pharmaceutically-acceptable salts of a selenium-based acids, and combinations thereof.

It will be appreciated by those of skill in the art that the uses, methods, and pharmaceutical compositions provided by the embodiments of the present invention encompass treatment of human and non-human subjects, research, diagnostic applications, and the like. Therefore, it is not intended that the present invention be limited to any subject and/or application situation.

Experimentation and Results

Animal tests were conducted to determine whether subject supplementation or intervention with the pharmaceutical compositions described herein might confer benefits like those associated with methionine restriction. The subject mice to be tested were fed an otherwise normal high-fat diet and supplemented with the first compounds and selenium compounds described herein as outline below. Various physiological parameters were observed and measured. The results show that the pharmaceutical compositions described herein confer a variety of health span benefits typically associated with dietary methionine restriction, including dramatically reduced white adipose tissue accumulation, improved glycemic control, and altered plasma levels of IGF-1, FGF-21, adiponectin, and leptin. These results indicate that the compositions and methods described herein produce benefits associated with methionine restriction, but in a normal, methionine-replete context.

All animal studies conducted were approved by the Institutional Animal Care and Use Committee (IACUC) of the Orentreich Foundation for the Advancement of Science, Inc. (Permit Number: 0511 MB). C57BL/6J mice (stock number 000664) were purchased from the Jackson Laboratories (Bar Harbor, Me.) and housed in a conventional animal facility maintained at 20±2° C., 50±10% relative humidity, with a 12 hr/12 hr light/dark photoperiod. Food and water were provided ad libitum. Upon arrival, mice were acclimatized for up to one week and fed Purina Lab Chow 5001 (Ralston Purina, Co.; St. Louis, Mo.).

Experiment 1—High-Fat Diet with Selenium Compounds

At the beginning of the experiment, the mice were divided into 5 groups with each group being fed one of five isocaloric (5.3 kcal/gm) high-fat diets ad libitum for several weeks. Each high-fat diet comprising a base of 12% kcal protein, 31% kcal carbohydrate, and 57% kcal fat. The first group's diet was the control-fed diet and included the base and methionine in an amount that represented about 0.86% of the weight of the food (sometimes referred to as “control group,” “control-fed group,” or “group 1”). The second group's diet was the methionine-restricted group where the base-diet was restricted to 0.12% methionine as a percentage of the total weight of the food (sometimes referred to as “methionine-restricted group” or “group 2”). The third group was fed the base diet containing 0.86% methionine, which was supplemented with sodium selenite in an amount representing 0.0073% of the total weight of the food (sometimes referred to as “sodium selenite-supplemented group” or “group 3”). The fourth group was fed the base diet containing 0.86% methionine, supplemented with 0.0036% selenomethionine, as a percentage of the total food weight (sometimes referred to as “group 4”). The fifth group was fed the base diet containing 0.86% methionine as a percentage of the total weight of the food, supplemented with 0.0073% selenomethionine as a percentage of the total weight of the food (sometimes referred to as “group 5”). Amounts of selenium-containing compounds used for this and subsequent experiments were determined from pilot studies aimed at identifying minimal doses that still achieve high efficacy. Full details concerning the compositions of the five diets are shown in the following Table 3. The sodium selenite diet group was observed for 13 weeks. The selenomethionine diet groups were observed for 16 weeks. The amount of selenomethionine administered to the animals in the fourth and fifth groups was approximately 1/200 and 1/100 times respectively, of the levels of sulfur-containing amino acids (i.e., methionine and cysteine) present in the subjects' diets.

TABLE 3

| | Diet 1 (CF) | | Diet 2 (MR) | | Diet 3 (CF-SS) | | Diet 4 (CF-SM) | | Diet 5 (CF-SM 2X) | |
|---|---|---|---|---|---|---|---|---|---|---|
| | g % | kcal % | g % | kcal % | g % | kcal % | g % | kcal % | g % | kcal % |
| Protein | 15 | 12 | 15 | 12 | 15 | 12 | 15 | 12 | 15 | 12 |
| Carbohydrate | 41 | 31 | 41 | 31 | 41 | 31 | 41 | 31 | 41 | 31 |
| Fat | 34 | 57 | 34 | 57 | 34 | 57 | 34 | 57 | 34 | 57 |
| Total | 100 | 100 | 100 | 100 | 100 | 100 | 100 | 100 | 100 | 100 |
| kcal/g | | 5.3 | | 5.3 | | 5.3 | | 5.3 | | 5.3 |
| Ingredient | g | kcal | g | kcal | g | kcal | g | kcal | g | kcal |
| L-Arginine | 11.2 | 45 | 11.2 | 45 | 11.2 | 45 | 11.2 | 45 | 11.2 | 45 |
| L-Histidine-HCl—H₂O | 3.3 | 13 | 3.3 | 13 | 3.3 | 13 | 3.3 | 13 | 3.3 | 13 |
| L-Isoleucine | 8.2 | 33 | 8.2 | 33 | 8.2 | 33 | 8.2 | 33 | 8.2 | 33 |
| L-Leucine | 11.1 | 44 | 11.1 | 44 | 11.1 | 44 | 11.1 | 44 | 11.1 | 44 |
| L-Lysine | 14.4 | 58 | 14.4 | 58 | 14.4 | 58 | 14.4 | 58 | 14.4 | 58 |
| DL-Methionine | 8.86 | 35 | 1.24 | 5 | 8.86 | 35 | 8.86 | 35 | 8.86 | 35 |
| L-Phenylalanine | 11.6 | 46 | 11.6 | 46 | 11.6 | 46 | 11.6 | 46 | 11.6 | 46 |
| L-Threonine | 8.2 | 33 | 8.2 | 33 | 8.2 | 33 | 8.2 | 33 | 8.2 | 33 |
| L-Tryptophan | 1.8 | 7 | 1.8 | 7 | 1.8 | 7 | 1.8 | 7 | 1.8 | 7 |
| L-Valine | 8.2 | 33 | 8.2 | 33 | 8.2 | 33 | 8.2 | 33 | 8.2 | 33 |
| L-Glutamic Acid | 27.83 | 111 | 35.5 | 142 | 27.83 | 111 | 27.83 | 111 | 27.83 | 111 |
| Glycine | 23.3 | 93 | 23.3 | 93 | 23.3 | 93 | 23.3 | 93 | 23.3 | 93 |
| L-Selenomethionine | 0 | 0 | 0 | 0 | 0 | 0 | 0.033 | 0 | 0.066 | 0 |
| Sodium Sdelenite | 0 | 0 | 0 | 0 | 0.066 | 0 | 0 | 0 | 0 | 0 |
| Corn Starch | 35 | 140 | 35 | 140 | 35 | 140 | 35 | 140 | 35 | 140 |
| Maltodextrin | 125 | 500 | 125 | 500 | 125 | 500 | 125 | 500 | 125 | 500 |
| Dextrose | 50 | 200 | 50 | 200 | 50 | 200 | 50 | 200 | 50 | 200 |
| Sucrose | 150 | 600 | 150 | 600 | 150 | 600 | 150 | 600 | 150 | 600 |
| Cellulose | 50 | 0 | 50 | 0 | 50 | 0 | 50 | 0 | 50 | 0 |
| Corn Oil | 46 | 414 | 46 | 414 | 46 | 414 | 46 | 414 | 46 | 414 |
| Lard | 257 | 2313 | 257 | 2313 | 257 | 2313 | 257 | 2313 | 257 | 2313 |
| Mineral Mix S10001 | 35 | 0 | 35 | 0 | 35 | 0 | 35 | 0 | 35 | 0 |
| Vitamin Mix V10001 | 10 | 40 | 10 | 40 | 10 | 40 | 10 | 40 | 10 | 40 |
| Choline Bitartrate | 2 | 0 | 2 | 0 | 2 | 0 | 2 | 0 | 2 | 0 |
| Dye | 0.05 | 0 | 0.05 | 0 | 0.05 | 0 | 0.05 | 0 | 0.05 | 0 |
| Total | 898 | 4759 | 898 | 4759 | 898 | 4759 | 898 | 4759 | 898 | 4759 |

Mice were randomly assigned to each of the diet groups such that each group had a similar average body mass (i.e., weight-matched). Body mass and food consumption were monitored once a week for sixteen weeks. Prior to blood collection, animals were fasted for 4 hours to establish a physiological baseline. Blood was then collected from the retro-orbital plexus and processed using EDTA-K2-coated blood collection tubes (Milian Dutscher Group; Geneva, Switzerland). The resulting plasma was frozen and stored at −80° C. until used for analysis. A portion of each blood sample was used for blood glucose determination using an Abbott Freestyle Lite glucometer and glucose strips (Abbott Diabetes Care, Inc.; Alameda, Calif.). At the end of each study, animals were fasted and bled, as described above. The mice were then sacrificed, their overall body condition was assessed, and certain physiological parameters were measured. The inguinal and perigonadal fat pads, as well as liver of each mouse, were harvested by surgical resection, weighed, flash frozen, and stored at −80° C.

Enzyme-linked immunosorbent assay (ELISA) kits were obtained commercially and used to measure plasma levels of IGF-1 (R&D Systems; Minneapolis, Minn.), adiponectin (R&D Systems), FGF-21 (Millipore Corp.; Billerica, Mass.), leptin (R&D Systems), growth hormone (Millipore Corp.), and insulin (ALPCO Diagnostics; Salem, N.H.). Tests were performed according to the manufacturers' recommendations and measured using a Molecular Devices SpectraMax M5 Microplate Reader (Molecular Devices LLC; San Jose, Calif.). Two technical replicates were performed for each sample and the statistical significance of the resulting values determined by unpaired two-tailed t-tests using the software package Prism 8 (Graph-Pad Software; La Jolla, Calif.).

Overall, it was found that the response to the selenomethionine-containing diets was similar to that of sodium selenite-containing diets, albeit somewhat less robust. For example, selenomethionine-supplemented male mice showed a dose-dependent protection against diet-induced obesity with less overall body mass than control-fed littermates as was the case for sodium selenite-supplemented animals, this reduction was primarily due to reduced adiposity, as less inguinal and perigonadal adipose tissue in selenomethionine-supplemented male mice was observed. However, this reduction did not extend to liver, as liver mass was not significantly different between experimental and control animals. It was confirmed that the moderate protection against diet-induced obesity observed was not due to calorie restriction, as animals ate the selenomethionine-containing diets comparably to the control diet, and their food consumption was actually somewhat higher than that of control-fed animals, when normalized for body mass.

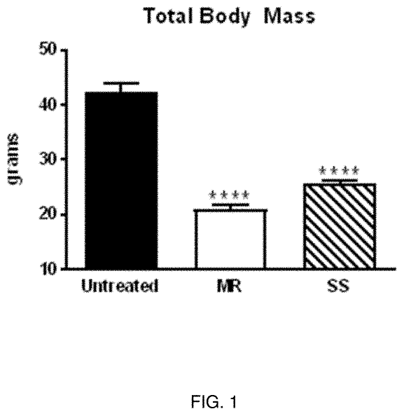

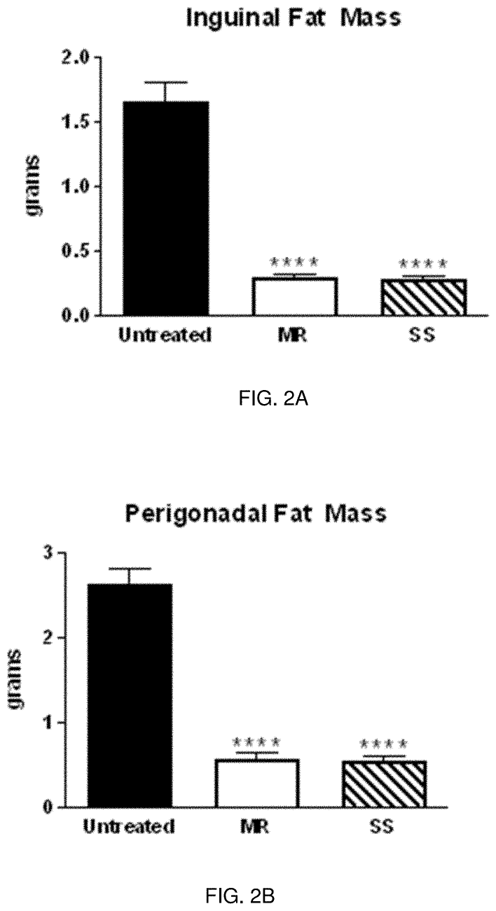

FIG. 1 is a graph showing the average mass of male mice that were administered selenium compound. In particular, the graph depicts the average mass of male mice fed the control-fed or untreated base diet, the average mass of the male mice fed the base diet supplemented with 0.0073% sodium selenite, and the average mass of the male mice fed the base diet with methionine dietarily restricted to 0.12% of the total weight of the food. The mass measurements were taken at the end of the experiment. Results shown are the averages of total body mass for 6 male mice for each feeding condition and error bars represent the standard error of the mean. Statistical significance was assessed using unpaired two-tailed t-tests and average body mass values that are significantly different from those of untreated control animals are indicated with asterisks (****, p<0.0001, where p is the p-value). For these experiments, methionine restriction and sodium selenite supplementation resulted in robust prevention of total weight gain over a 16 week interval in the context of a high-fat diet, wherein 57% of calories were supplied from fat. The observed prevention of weight gain was due primarily to the reduced accumulation of white adipose tissue engendered by these interventions (see FIGS. 2A and 2B, below). In addition, similar results were obtained for females, as well as animals (both male and female) supplemented with the organoselenium compound selenomethionine. All such results are summarized in Table 2 above. It should be noted by comparing the methionine restriction group results to the control-fed group results that reduced body mass is a methionine restriction benefit. The results shown by the bar graph, along with the Table 2 summary, clearly show that supplementation with selenium such as sodium selenite and selenomethionine provides such a benefit.

FIGS. 2A and 2B show bar graphs depicting white adipose tissue accumulation of male mice that were administered selenium compound. In particular, the graphs depict the average mass of fat pads surgically resected from the inguinal (FIG. 2A) and perigonadal (FIG. 2B) depots of mice fed the normal (untreated) and altered (SS, 0.0073% sodium selenite; MR, 0.12% methionine) diets at the end of the experiment. Results shown are averages of fat pad mass for 6 mice for each feeding condition and error bars represent the standard error of the mean. Statistical significance was assessed using unpaired two-tailed t-tests and average fat pad mass vales that are significantly different from those of untreated control animals are indicated with asterisks (****, p<0.0001). For these experiments, methionine restriction and sodium selenite supplementation resulted in robust protection against the accumulation of adipose tissue over a 16 week interval in the context of a high-fat diet, wherein 57% of calories were supplied from fat. Similar results were obtained for females, as well as animals (both male and female) supplemented with the organoselenium compound selenomethionine. All such results are summarized in Table 2 above. It should be noted by comparing the methionine restriction group results to the control-fed group results that reduced white adipose tissue accumulation is a methionine restriction benefit. The results shown by the bar graph, along with the Table 2 summary, clearly show that supplementation with selenium such as sodium selenite and selenomethionine provides such a benefit.

FIGS. 1, 2A, and 2B along with Table 2 above, demonstrate that selenium supplementation protects mice against the dramatic weight gain observed for control-fed animals. In fact, the protection against diet-induced obesity conferred by sodium selenite-supplementation was essentially identical to that observed for methionine-restricted mice. In addition, selenium supplementation resulted in a reduction in the accumulation of both inguinal and perigonadal adipose tissue. To confirm that the observed differences in fat accumulation and body condition was due directly to the presence of the compound rather than a putative calorie reduction in the event that animals found the food unpalatable, the rate of food consumption for the selenium-supplemented diet as well as the control-fed and methionine-restricted diets were assessed. With respect to absolute food consumption, sodium selenite-containing food was consumed equivalently to the control diet. Thus, mice found sodium selenite-containing food to be just as palatable as the control diet and were not calorie-restricted. This confirms that the total protection against diet-induced obesity enjoyed by mice was due to selenium supplementation.

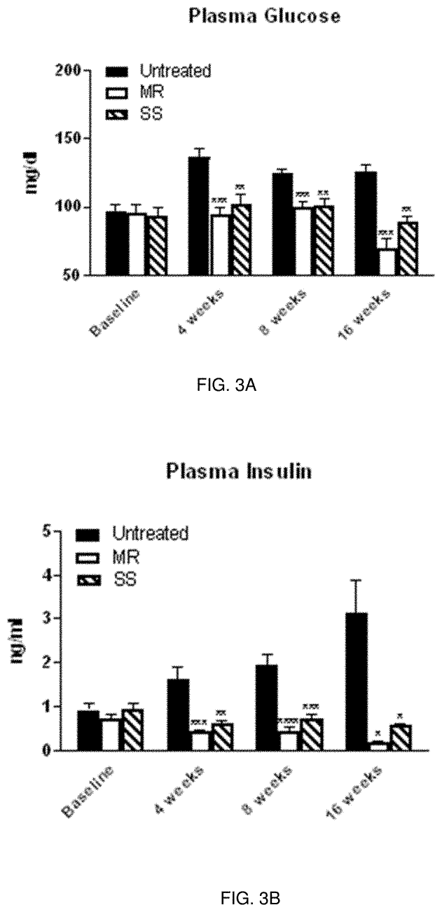

FIGS. 3A and 3B show bar graphs depicting the longitudinal maintenance of glucose and insulin homeostasis in male mice supplements with selenium compound. In particular, the graphs depict the average plasma levels of glucose (FIG. 3A) and insulin (FIG. 3B) for mice fed the control-fed or untreated base diet, the average plasma levels of glucose (FIG. 3A) and insulin (FIG. 3B) for mice fed the base diet supplemented with 0.0073% sodium selenite as a percentage of total food mass or weight, and the average plasma levels of glucose (FIG. 3A) and insulin (FIG. 3B) for mice fed the base diet with methionine dietarily restricted to 0.12% of total food by weight. Results shown are averages of plasma glucose and insulin levels for 6 mice for each feeding condition and error bars represent the standard error of the mean. Statistical significance was assessed using unpaired two-tailed t-tests and average plasma analyte vales that are significantly different from those of untreated control animals are indicated with asterisks (*, p<0.05; **, p<0.01; ***, p<0.001; ****, p<0.0001). For these experiments, methionine restriction and sodium selenite supplementation resulted in maintenance of low plasma glucose and insulin levels over a 16 week interval in the context of a high-fat diet, wherein 57% of calories were supplied from fat. Similar results were obtained for females. All such results are summarized in Table 2 above. It will be seen by comparing the methionine restriction group results to the control-fed or “untreated” group results that reduced plasma glucose and insulin levels are methionine restriction benefits. The results shown by the bar graph, along with the Table 2 summary clearly show that supplementation with selenium such as sodium selenite and selenomethionine provides such a benefit.

In order to determine whether selenium supplementation shows downregulated IGF-1 signaling in the form of decreased plasma IGF-1 levels in C57BL/6J mice as compared to control groups and dietarily restricted methionine groups, multiple circulating analytes from male mice fed the control (group 1), methionine-restricted (group 2), and sodium selenite-supplemented (group 3) diets were assessed. For this purpose, blood samples were obtained from animals at baseline (i.e., before initiation of the experimental diets), as well as after 4 weeks, 8 weeks, and 16 weeks on diet (with the last time-point representing the conclusion of the experiment). Plasma samples were analyzed by ELISAs to determine the concentrations of IGF-1, FGF-21, leptin, and adiponectin.

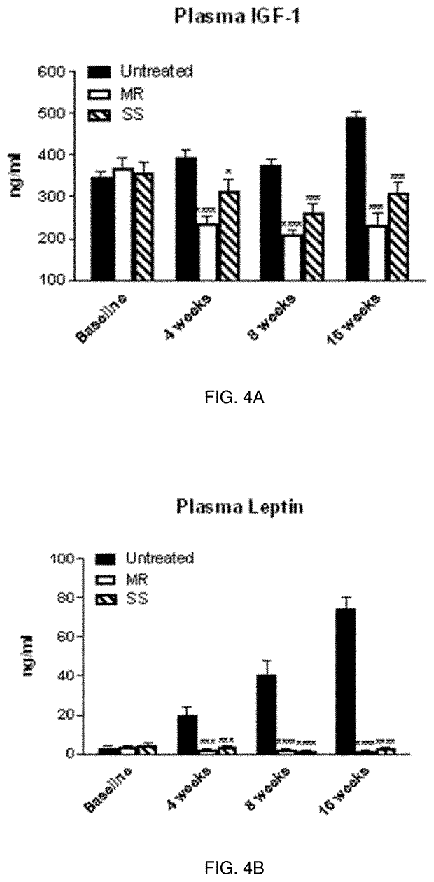

FIGS. 4A and 4B show bar graphs depicting the longitudinal maintenance of low IGF-1 and leptin levels in male mice fed the third group diet. In particular, the graphs depict the average plasma levels of IGF-1 (FIG. 4A) and leptin (FIG. 4B) for mice fed the control-fed or untreated base diet, the average plasma levels of IGF-1 (FIG. 4A) and leptin (FIG. 4B) for mice fed the base diet supplemented with 0.0073% sodium selenite, and the average plasma levels of IGF-1 (FIG. 4A) and leptin (FIG. 4B) for mice fed the base diet with methionine dietarily restricted to 0.12% of total food weight. Results shown are averages of plasma IGF-1 and leptin levels for 6 mice for each feeding condition and error bars represent the standard error of the mean. Statistical significance was assessed using unpaired two-tailed t-tests and average plasma analyte vales that are significantly different from those of untreated control animals are indicated with asterisks (*, p<0.05; ***, p<0.001; ****, p<0.0001). For these experiments, methionine restriction and sodium selenite supplementation resulted in sustained reductions of plasma IGF-1 levels, as well as maintenance of low plasma leptin levels, over a 16 week interval in the context of a high-fat diet, wherein 57% of calories were supplied from fat. Similar results were obtained for females. All such results are summarized in Table 2 above. It will be seen by comparing the methionine restriction group results to the control-fed or “untreated” group results that reduced plasma IGF-1 and leptin levels are methionine restriction benefits. The results shown by the bar graph, along with the Table 2 summary clearly show that supplementation with selenium such as sodium selenite and selenomethionine provides such a benefit.

FIGS. 3A, 3B, 4A, and 4B, along with Table 1 above indicate that supplementation with selenium provided downregulation of IGF-1 as well as glycemic regulation. At the conclusion of experiment 1, it can be seen that plasma IGF-1 levels were reduced by 37% for sodium selenite-supplemented mice as compared with control values. Similar reductions were seen at all time-points following initiation of feeding the experimental diets. Plasma levels of leptin followed a similar trend. Male mice that had been fed sodium selenite-supplemented diets for 16 weeks showed reductions in circulating leptin of 96% as compared with controls. Plasma glucose and insulin levels were also reduced in group 3, which was fed the sodium selenite supplemented diet, as compared to the control group. This finding suggests that the beneficial metabolic milieu conferred to male mice by selenium supplementation also includes improved glycemic control.

Experiment 2—High-Fat Diet with First Compounds

At the beginning of this experiment, the mice were divided into 5 groups with each group being fed one of five isocaloric (5.3 kcal/gm) high-fat diets ad libitum for 9 weeks, with each diet comprising a base of 12% kcal protein, 31% kcal carbohydrate, and 57% kcal fat. The first group's diet was the control-fed diet and included the base and 0.86% methionine as a percentage of total food weight (sometimes referred to as “control group,” “control-fed group,” or “group 1”). The second group's diet was the methionine-restricted group where the base-diet was restricted to 0.12% methionine as a percentage of total food weight (sometimes referred to as “methionine-restricted group” or “group 2”). The third group was fed the base diet containing 0.86% methionine, which was supplemented with lysine (a first compound) in an amount representing 6.45% of the total food consumed by this group (sometimes referred to as “lysine-supplemented group,” “LYS group,” or “group 3”). The fourth group was fed the base diet containing 0.86% methionine, supplemented with O-benzyl serine (a first compound) in an amount representing 4.3% of the total food consumed by this group (sometimes referred to as the “OBS-supplemented group,” “OBS group” or “group 4”). The fifth group was fed the base diet containing 0.86% methionine, supplemented with S-phenyl cysteine (a first compound) in amount representing 4.3% of the total food consumed by this group (sometimes referred to as the “SPC-supplemented group,” “SPC group,” or “group 5”). Amounts of first compounds used for this and subsequent experiments were determined from pilot studies aimed at identifying minimal doses that still achieve high efficacy.

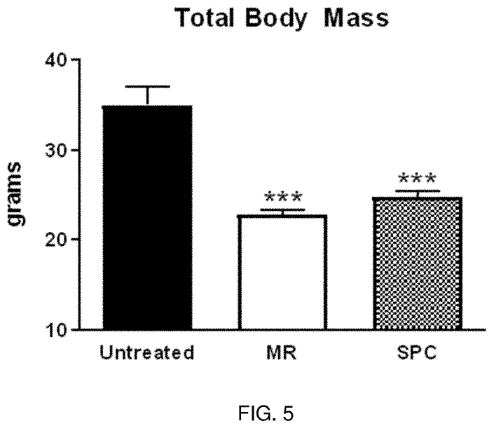

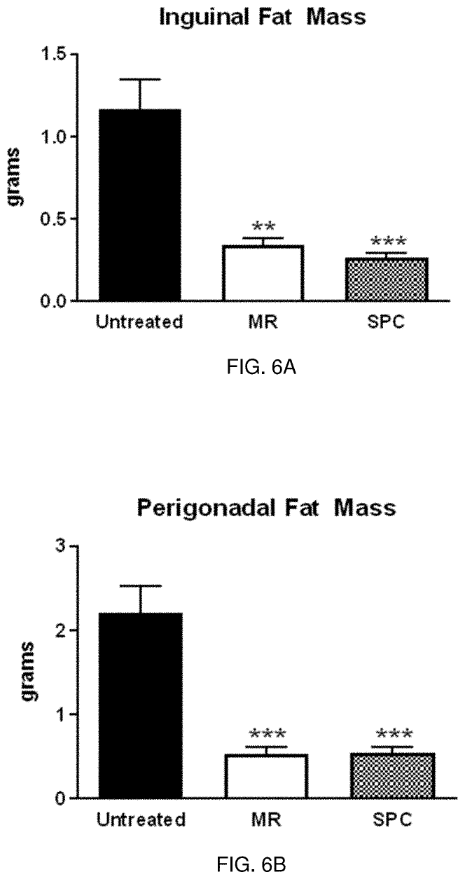

FIG. 5 is a graph showing the average body mass of female mice fed the group 5 diet as compared to the control group diet and the dietarily restricted methionine group diet. In particular, the graph depicts the average mass of mice fed the normal (untreated) and altered (SPC, 4.3% S-phenyl cysteine; MR, 0.12% methionine) diets at the end of the experiment. Results shown are averages of total body mass for 8 mice for each feeding condition and error bars represent the standard error of the mean. Statistical significance was assessed using unpaired two-tailed t-tests and average body mass values that are significantly different from those of untreated control animals are indicated with asterisks (***, p<0.001). For these experiments, methionine restriction and S-phenyl cysteine supplementation resulted in robust prevention of total weight gain over a 9 week interval in the context of a high-fat diet, wherein 57% of calories were supplied from fat. The observed prevention of weight gain was due primarily to the reduced accumulation of white adipose tissue engendered by these interventions (see FIGS. 6A and 6B, below). In addition, similar results were obtained for males, as well as animals supplemented with the compounds lysine, O-benzyl serine, and tryptophan. All such results are summarized in Table 1 above. The amount of these compounds administered to the animals was about 5 to about 7.5 times greater than the levels of sulfur-containing amino acids (i.e., methionine and cysteine) present in their diets. Comparing the results of the control-fed group and the methionine-restricted group indicated that reduced body mass is a methionine restriction benefit. The graph results along with the Table 1 summary also clearly indicate that supplementation with a first compound such as lysine, O-benzyl serine, tryptophan, and S-phenyl cysteine provides such a benefit.

FIGS. 6A and 6B are graphs showing white adipose tissue accumulation for female mice fed the group 5 diet as compared to the control group and the dietarily restricted methionine group. In particular, the graphs depict the average mass of fat pads surgically resected from the inguinal (FIG. 6A) and perigonadal (FIG. 6B) depots of mice fed the normal (untreated) and altered (SPC, 4.3% S-phenyl cysteine; MR, 0.12% methionine) diets at the end of the experiment. Results shown are averages of fat pad mass for 8 mice for each feeding condition and error bars represent the standard error of the mean. Statistical significance was assessed using unpaired two-tailed t-tests and average fat pad mass vales that are significantly different from those of untreated control animals are indicated with asterisks (***, p<0.001; ***, p<0.001). For these experiments, methionine restriction and S-phenyl cysteine supplementation resulted in robust protection against the accumulation of adipose tissue over a 9 week interval in the context of a high-fat diet, wherein 57% of calories were supplied from fat. Similar results were obtained for males, as well as animals supplemented with the compounds lysine, O-benzyl serine, and tryptophan. All such results are summarized in Table 1 above. The amount of the indicated first compounds administered to the animals was about 5 to about 7.5 times greater than the levels of sulfur-containing amino acids (i.e., methionine and cysteine) present in their diets. Comparing the results of the control-fed group and the methionine-restricted group indicates that reduced white adipose tissue accumulation is a methionine restriction benefit. The graph results and Table 1 summary also clearly indicate that supplementation with a first compound such as lysine, O-benzyl serine, tryptophan, and S-phenyl cysteine provides such a benefit.

FIGS. 5, 6A, and 6B, along with Table 1 above, demonstrate that supplementation with a first compound protects mice against the dramatic weight gain observed for control-fed animals. First compound supplementation resulted in a reduction in body mass, as well as the accumulation of both inguinal and perigonadal adipose tissue. To confirm that the observed differences in fat accumulation and body condition was due directly to the presence of the compound rather than a putative calorie reduction in the event that animals found the food unpalatable, the rate of food consumption for the first compound-supplemented diet as well as the control-fed and methionine-restricted diets were assessed. With respect to absolute food consumption, first compound-containing food was consumed equivalently to the control diet. Thus, mice found first compound-containing food to be just as palatable as the control diet and were not calorie-restricted. This confirms that the total protection against diet-induced obesity enjoyed by mice was due to first compound supplementation.

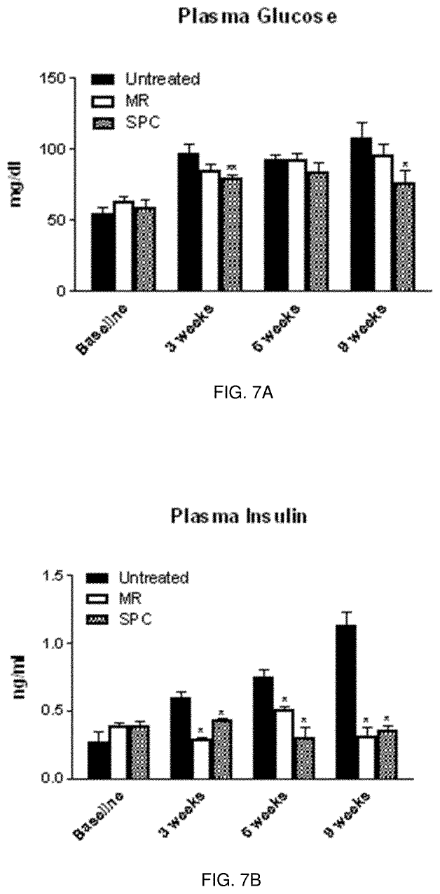

FIGS. 7A and 7B are bar graphs depicting the longitudinal maintenance of glucose and insulin homeostasis in female mice supplemented with S-phenyl cysteine (SPC) as compared to methionine-restricted (MR) littermates and the control-fed or “untreated” animals. In particular, the graphs depict the average plasma levels of glucose (FIG. 7A) and insulin (FIG. 7B) for mice fed the normal (untreated) and altered (SPC, 4.3% S-phenyl cysteine; MR, 0.12% methionine) diets over the course of the experiment. Results shown are averages of plasma glucose and insulin levels for 8 mice for each feeding condition and error bars represent the standard error of the mean. Statistical significance was assessed using unpaired two-tailed t-tests and average plasma analyte vales that are significantly different from those of untreated control animals are indicated with asterisks (*, p<0.05; **, p<0.01). For these experiments, S-phenyl cysteine supplementation resulted in a maintenance of low plasma glucose (making it more efficacious than methionine restriction in this case) and both methionine restriction and S-phenyl cysteine supplementation resulted in maintenance of low insulin levels. This occurred over a 9 week interval in the context of a high-fat diet, wherein 57% of calories were supplied from fat. Similar results were obtained for males, as well as animals supplemented with the compounds lysine, O-benzyl serine, and tryptophan. All such results are summarized in Table 1 above. Comparing the results of the control-fed group and the methionine-restricted group indicated that reduced body mass is a methionine restriction benefit. The graph results and table 1 summary also clearly indicate that supplementation with a first compound such as lysine, O-benzyl serine, tryptophan, and S-phenyl cysteine provides such a benefit.

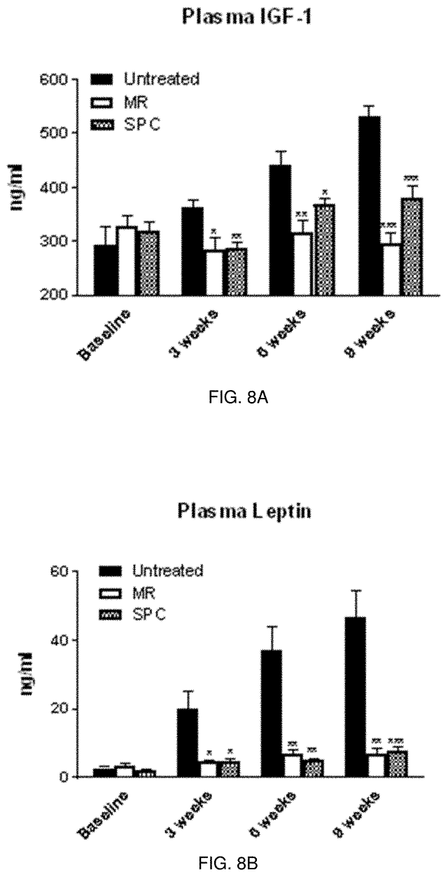

FIGS. 8A and 8B depict the longitudinal maintenance of low IGF-1 and leptin levels in female mice supplemented with S-phenyl cysteine (SPC) and methionine-restricted (MR) littermates as compared with untreated control animals. In particular, the graphs depict the average plasma levels of IGF-1 (FIG. 8A) and leptin (FIG. 8B) for mice fed the normal (untreated) and altered (SPC, 4.3% S-phenyl cysteine; MR, 0.12% methionine) diets over the course of the experiment. Results shown are averages of plasma IGF-1 and leptin levels for 8 mice for each feeding condition and error bars represent the standard error of the mean. Statistical significance was assessed using unpaired two-tailed t-tests and average plasma analyte vales that are significantly different from those of untreated control animals are indicated with asterisks (*, p<0.05; **, p<0.01; ***, p<0.001). For these experiments, both methionine restriction and S-phenyl cysteine supplementation resulted in sustained reductions of plasma IGF-1 levels, as well as maintenance of low plasma leptin levels, over a 9 week interval in the context of a high-fat diet, wherein 57% of calories were supplied from fat. Similar results were obtained for males, as well as animals supplemented with the compounds lysine, O-benzyl serine, and tryptophan. All such results are summarized in Table 1 above. Comparing the results of the control-fed group and the methionine-restricted group indicated that reduced body mass is a methionine restriction benefit. The graph results, as well as the Table 1 summary, clearly indicate that supplementation with a first compound such as lysine, O-benzyl serine, tryptophan, and S-phenyl cysteine provide such a benefit.

FIGS. 7A, 7B, 8A, and 8B, along with Table 1 above, show that supplementation with a first compound provides downregulation of IGF-1 as well as glycemic regulation. At the conclusion of experiment 2, it can be seen that plasma IGF-1 levels were reduced significantly in supplemented mice as compared with control values. Plasma levels of leptin followed a similar trend. Plasma glucose and insulin levels were also reduced. This finding suggests that the beneficial metabolic milieu conferred to female mice by selenium supplementation also includes improved glycemic control.

Experiment 3—Low-Fat Diet with Selenium Compound Supplementation

In this experiment, male mice were divided into three groups, with each group being fed a separate low-fat diet ad libitum for 8 weeks. The first group represented the control-fed group (CF group) and was fed a low-fat base diet that included 0.86% methionine as a percentage of total food. The second group was fed a methionine-restricted diet (MR group) where the amount of methionine was restricted to 0.12% of total consumed. The third group was fed the base diet supplemented with selenomethionine (SMS group) in an amount representing 0.00015% of total food consumed. Full details concerning the compositions of the three diets are shown in the following Table 4.

TABLE 4

| | Dial 1 (CF) | | Diet 2 (MR) | | Diet 3 (CF-SMS) | |
|---|---|---|---|---|---|---|
| | g % | kcal % | g % | kcal % | g % | kcal % |
| Protein | 13 | 14 | 13 | 14 | 13 | 14 |
| Carbohydrate | 74 | 76 | 74 | 76 | 74 | 76 |
| Fat | 4 | 10 | 4 | 10 | 4 | 10 |
| Total | 100 | 100 | 100 | 100 | 100 | 100 |
| kcal/g | | 3.9 | | 3.9 | | 3.9 |
| Ingredient | g | kcal | g | kcal | g | kcal |
| L-Arginine | 11.2 | 45 | 11.2 | 45 | 11.2 | 45 |
| L-Histidine-HCl—H₂O | 3.3 | 13 | 3.3 | 13 | 3.3 | 13 |
| L-Isoleucine | 8.2 | 33 | 8.2 | 33 | 8.2 | 33 |
| L-Leucine | 11.1 | 44 | 11.1 | 44 | 11.1 | 44 |
| L-Lysine | 14.4 | 58 | 14.4 | 58 | 14.4 | 58 |
| DL- Methionine | 8.86 | 35 | 1.24 | 5 | 8.86 | 35 |
| L-Phenylalanine | 11.6 | 46 | 11.6 | 46 | 11.6 | 46 |
| L-Threonine | 8.2 | 33 | 8.2 | 33 | 8.2 | 33 |
| l-Tryptophan | 1.8 | 7 | 1.8 | 7 | 1.8 | 7 |
| L-Valine | 8.2 | 33 | 8.2 | 33 | 8.2 | 33 |
| L-Glutamic Acid | 27.83 | 111 | 35.5 | 142 | 27.83 | 111 |
| Glycine | 23.3 | 93 | 23.3 | 93 | 23.3 | 93 |
| L-Selenomethionine | 0 | 0 | 0 | 0 | 0.00148 | 0 |
| Sodium Selenite | 0 | 0 | 0 | 0 | 0 | 0 |
| Corn Starch | 424.5 | 1698 | 424.5 | 1698 | 424.5 | 1698 |
| Maltodextrin | 125 | 500 | 125 | 500 | 125 | 500 |
| Dextrose | 50 | 200 | 50 | 200 | 50 | 200 |
| Sucrose | 150 | 600 | 150 | 600 | 150 | 600 |
| Cellulose | 50 | 0 | 50 | 0 | 50 | 0 |
| Corn Oil | 46 | 414 | 46 | 414 | 46 | 414 |
| Lard | 0 | 0 | 0 | 0 | 0 | 0 |
| Mineral Mix S10001 | 35 | 0 | 35 | 0 | 35 | 0 |
| Vitamin Mix V10001 | 10 | 40 | 10 | 40 | 10 | 40 |
| Choline Bitartrate | 2 | 0 | 2 | 0 | 2 | 0 |
| Dye | 0.05 | 0 | 0.05 | 0 | 0.05 | 0 |
| Total | 1031 | 4004 | 1031 | 4004 | 1031 | 4004 |

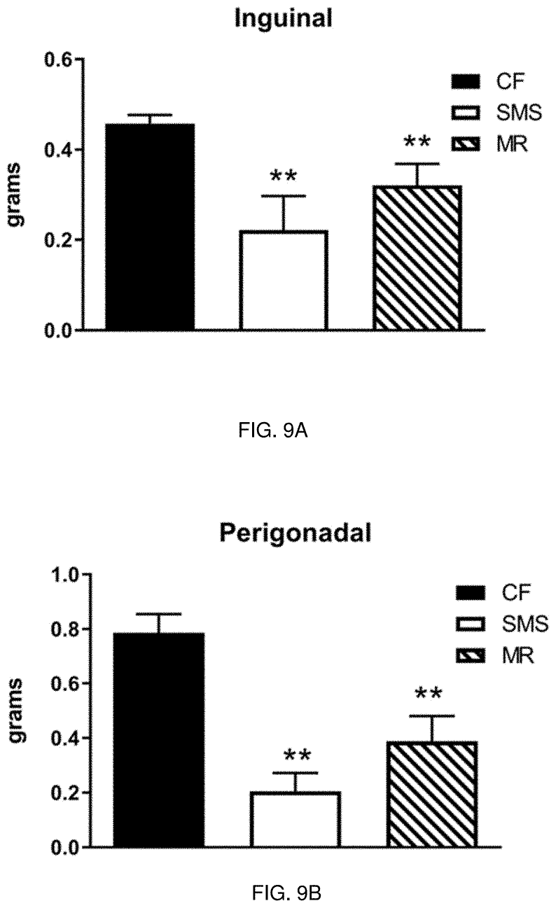

FIGS. 9A and 9B depict the reduced white adipose tissue accumulation of the SMS group as compared with the MR group and the CF group. In particular, the graph depicts the average weight of fat pads surgically resected from the inguinal (FIG. 9A) and perigonadal (FIG. 9B) depots of mice fed the indicated diets (selenomethionine-supplemented, SMS; control-fed, CF; and methionine-restricted, MR). Results shown are averages of fat pad weights for 4 mice for each feeding condition and error bars represent the standard error of the mean. Statistical significance was assessed using unpaired two-tailed t-tests, and fat pad average weights that are significantly different from those of control-fed animals are indicated with asterisks (**p<0.01). For these experiments, selenomethionine supplementation clearly resulted in a robust protection against the accumulation of adipose tissue over an 8 week interval and did so even more effectively than methionine restriction. This protection occurred in the context of an otherwise standard mouse diet, wherein 10% of calories were supplied from fat. In addition, the amount of selenomethionine administered to the animals was approximately 1/6000 that of the sulfur-containing amino acids (i.e., methionine and cysteine) present in their diets. It should be noted by comparing the methionine-restricted group results to the control-fed group results that reduced white adipose tissue accumulation is a methionine restriction benefit. The results shown by the bar graphs of FIGS. 9A and 9B clearly indicate that supplementation with a selenium compound such as selenomethionine provides such a benefit, even in the context of a low-fat diet.

The scope of the present invention is defined by the appended claims.

What is claimed:

1. A pharmaceutical composition for providing a benefit associated with a methionine-restricted diet to a subject without restricting the subject's methionine consumption, the pharmaceutical composition comprising:
one or more of a first compound and a selenium compound, wherein the first compound comprises at least one compound chosen from lysine, O-benzyl serine (OBS), S-phenyl cysteine (SPC), S-benzyl cysteine (SBC), isoleucine, taurine, tryptophan, and 2-aminobicyclo-(2.2.1)-heptane-2-carboxylic acid (BCH).

2. The pharmaceutical composition of claim 1, wherein the selenium compound comprises organic selenium.

3. The pharmaceutical composition of claim 2, wherein the selenium compound comprises at least one compound chosen from selenomethionine, purified selenomethionine, 1,4-phenylenebis(methylene)selenocyanate, selenohomocysteine, selenocystathionine, selenocysteine/selenocystine, selenoglutathione trisulfide, methaneselenol, dimethyl selenide, trimethyl selenide, and selenized yeast extract.

4. The pharmaceutical composition of claim 1, wherein the selenium compound comprises inorganic selenium.

5. The pharmaceutical composition of claim 4, wherein selenium compound comprises a pharmaceutically-acceptable salt of a selenium-based acid.

6. The pharmaceutical composition of claim 5, wherein selenium compound comprises sodium selenite.

7. The pharmaceutical composition of claim 1, wherein the pharmaceutical composition comprises a first compound comprising at least about 2.5 times the amount of sulfur amino acids in the subject's diet by weight.

8. The pharmaceutical composition of claim 7, wherein the pharmaceutical composition comprises a first compound comprising at least about 5 times the amount of sulfur amino acids in the subject's diet by weight.

9. The pharmaceutical composition of claim 1, wherein the pharmaceutical composition comprises a first compound comprising at least about 1.25 grams

10. The pharmaceutical composition of claim 1, wherein the pharmaceutical composition comprises a first compound comprising at least about 4.3% of food consumed by the subject by weight.

11. The pharmaceutical composition of claim 1 wherein the pharmaceutical composition comprises an effective amount of selenium compound comprising at least about 1/6000 times the amount of sulfur amino acids consumed in the subject's diet.

12. The pharmaceutical composition of claim 1 wherein the pharmaceutical composition comprises an effective amount of selenium compound comprising at least about 0.0833 mg.

13. The pharmaceutical composition of claim 1 wherein the pharmaceutical composition comprises an effective amount of selenium compound comprising comprises at least about 0.00015% of food consumed by the subject by weight.

14. The pharmaceutical composition of claim 1, wherein the pharmaceutical composition comprises at least one of a first compound and a selenium compound in an amount such that administration of the pharmaceutical composition to a subject, causes the subject to experience one or more of reduced body mass, reduced inguinal fat mass, reduced perigonadal fat mass, reduced plasma glucose, reduced plasma insulin, reduced plasma IGF-1, reduced plasma leptin, increased plasma adiponectin and increased plasma FGF-21, as compared to a subject not treated with the pharmaceutical composition.

15. A method of providing a benefit associated with a methionine-restricted diet to a subject without restricting the subject's methionine consumption, the method comprising:
administering pharmaceutical composition to the subject, the pharmaceutical composition comprising an effective amount of one or more of a first compound and a selenium compound, wherein the first compound comprises at least one compound chosen from lysine, O-benzyl serine (OBS), S-phenyl cysteine (SPC), S-benzyl cysteine (SBC), isoleucine, taurine, tryptophan, and 2-aminobicyclo-(2.2.1)-heptane-2-carboxylic acid (BCH).

16. The method of claim 15, wherein the methionine restriction benefit comprises at least one benefit chosen from reduced adipose tissue accumulation, reduced body mass, reduced fatty liver, reduced circulating levels of IGF-1, increased circulating levels of FGF-21, reduced circulating levels of leptin, increased circulating levels of adiponectin, reduced circulating levels of glucose, and reduced circulating levels of insulin.

17. The method of claim 15, wherein the selenium compound comprises at least one compound chosen from selenomethionine, 1,4-phenylenebis (methylene) selenocyanate, selenohomocysteine, selenocystathionine, selenocysteine/selenocystine, selenoglutathione trisulfide, methaneselenol, dimethyl selenide, trimethyl selenide, purified selenomethionine, selenized yeast extract, and pharmaceutically-acceptable salts of selenium-based acids.

18. The method of claim 15, wherein an effective amount of a first compound comprises at least about 2.5 times the amount of sulfur amino acids in the subject's diet by weight.

19. The method of claim 15, wherein an effective amount of a first compound comprises at least about 1.25 grams.

20. The method of claim 15, wherein an effective amount of a first compound comprises at least about 4.3% of food consumed by the subject by weight.

21. The method of claim 15, wherein an effective amount of selenium compound comprises at least about 1/6000 times the amount of sulfur amino acids consumed in the subject's diet.

22. The method of claim 15, wherein an effective amount of selenium compound comprises at least about 0.0833 mg.

23. The method of claim 15, wherein an effective amount of selenium compound comprises at least about 0.00015% of food consumed by the subject by weight.

24. The method of claim 15, wherein an effective amount of one or more of a first compound and a selenium compound comprises an amount that causes the subject to accumulate at least about 9% less body as compared to an untreated subject.

25. The method of claim 15, wherein an effective amount of one or more of a first compound and a selenium compound comprises an amount that causes the treated subject to accumulate at least about 40% less white adipose tissue as compared to an untreated subject.

26. The method of claim 15, wherein the effective amount of one or more of a first compound and a selenium compound comprises an amount that causes the treated subject to accumulate at least about 6% less plasma glucose as compared to an untreated subject.

27. The method of claim 15, wherein the effective amount of one or more of a first compound and a selenium compound comprises an amount that causes the treated subject to accumulate at least about 44% less plasma insulin as compared to an untreated subject.

28. The method of claim 15, wherein the effective amount of one or more of a first compound and a selenium compound comprises an amount that causes the treated subject to accumulate at least about 21% less plasma TGF-1 as compared to an untreated subject.

29. The method of claim 15, wherein the effective amount of a of one or more of a first compound and a selenium compound comprises an amount that causes the treated subject to accumulate at least about 72% less plasma leptin as compared to an untreated subject.

30. The method of claim 15, wherein the effective amount of one or more of a first compound and a selenium compound comprises an amount that causes the treated subject to accumulate at least about 6% more plasma adiponectin as compared to an untreated subject.

31. The method of claim 15, wherein the effective amount of one or more of a first compound and a selenium compound comprises an amount that causes the treated subject to accumulate at least about 44% more plasma FGF-21 as compared to an untreated subject.

32. A method of downregulating IGF-1 signaling in a subject, the method comprising administering a pharmaceutical composition to the subject, the pharmaceutical composition comprising an effective amount of one or more of a first compound and a selenium compound, wherein the first compound comprises at least one compound chosen from lysine, O-benzyl serine (OBS), S-phenyl cysteine (SPC), S-benzyl cysteine (SBC), isoleucine, taurine, tryptophan, and 2-aminobicyclo-(2.2.1)-heptane-2-carboxylic acid (BCH), and wherein the selenium compound comprises at least one compound chosen from selenomethionine, 1,4-phenylenebis (methylene) selenocyanate, selenohomocysteine, selenocystathionine, selenocysteine/selenocystine, selenoglutathione trisulfide, methaneselenol, dimethyl selenide, trimethyl selenide, purified selenomethionine, selenized yeast extract, and pharmaceutically-acceptable salts of selenium-based acids.

33. A method of treating a disease or disorder associated with diet-induced obesity, low metabolism, hyperinsulinemia, adipose tissue accumulation, fatty liver, or combinations thereof in a subject, the method comprising administering a pharmaceutical composition to the subject, the pharmaceutical composition comprising an effective amount of one or more of a first compound and a selenium compound, wherein the first compound comprises at least one compound chosen from lysine, O-benzyl serine (OBS), S-phenyl cysteine (SPC), S-benzyl cysteine (SBC), isoleucine, taurine, tryptophan, and 2-aminobicyclo-(2.2.1)-heptane-2-carboxylic acid (BCH), and wherein the selenium compound comprises at least one compound chosen from selenomethionine, 1,4-phenylenebis (methylene) selenocyanate, selenohomocysteine, selenocystathionine, selenocysteine/selenocystine, selenoglutathione trisulfide, methaneselenol, dimethyl selenide, trimethyl selenide, purified selenomethionine, selenized yeast extract, and pharmaceutically-acceptable salts of selenium-based acids.

34. The use of a pharmaceutical composition comprising at least one of a first compound and a selenium compound in the manufacture of a medicament for providing a benefit associated with a methionine-restricted diet to a subject without restricting the subject's methionine consumption, wherein the first compound comprises at least one compound chosen from lysine, O-benzyl serine (OBS), S-phenyl cysteine (SPC), S-benzyl cysteine (SBC), isoleucine, taurine, tryptophan, and 2-aminobicyclo-(2.2.1)-heptane-2-carboxylic acid (BCH), and wherein the selenium compound comprises at least one compound chosen from selenomethionine, selenohomocysteine, selenocystathionine, selenocysteine/selenocystine, selenoglutathione trisulfide, methaneselenol, dimethyl selenide, trimethyl selenide, purified selenomethionine, selenized yeast extract, and pharmaceutically-acceptable salts of selenium-based acids.
